(19) United States
(12) Patent Application Publication
(10) Pub. No.: US 2026/0114785 A1
(43) Pub. Date: Apr. 30, 2026

(54) TILE-BASED NEURAL RECORDING AND STIMULATION SYSTEM

(71) Applicant: Blackrock Microsystems, Inc., Salt Lake City, UT (US)

(72) Inventors: Florian SOLZBACHER, Salt Lake City, UT (US); Pierre-Emmanuel GAILLARDON, Salt Lake City, UT (US); Matthieu Arnaud COURIOL, Salt Lake City, UT (US); Seyed Mohammad Ali ZEINOLABEDIN, Salt Lake City, UT (US); Moritz Michael LEBER, Salt Lake City, UT (US); Daniel Richard POWELL, Salt Lake City, UT (US); Robert Kyle FRANKLIN, IV, Centerville, UT (US)

(21) Appl. No.: 19/372,068

(22) Filed: Oct. 28, 2025

Related U.S. Application Data
(60) Provisional application No. 63/713,536, filed on Oct. 29, 2024.

(51) Int. Cl. A61B 5/37; A61B 5/268; A61B 5/271; A61B 5/293; A61B 5/383

(52) U.S. Cl. CPC A61B 5/37; A61B 5/268; A61B 5/271; A61B 5/293; A61B 5/383

(57) ABSTRACT

In some embodiments, an apparatus comprises a biocompatible substrate configured to be disposed on a surface of a brain and including a plurality of nodes and a plurality of connectors interconnecting the plurality of nodes, the biocompatible substrate defining a plurality of openings therein, each of the plurality of nodes and the plurality of connectors including at least one encapsulation material. The plurality of nodes including: a first set of nodes, each node from the first set of nodes including (i) an associated set of electrodes and (ii) an associated analog circuit; a second set of nodes different than the first set of nodes, each node from the second set of nodes including a digital circuit; and a third set of nodes different than the first and second set of nodes, the third set of nodes configured to supply power to the first and second set of nodes.

### CROSS-REFERENCE TO RELATED APPLICATIONS

This application claims priority to and the benefit of U.S. Provisional Application No. 63/713,536 filed Oct. 29, 2024, and titled, “Tile-Based Neural Recording and Stimulation System,” the disclosure of which is incorporated by reference herein in its entirety.

### TECHNICAL FIELD

Embodiments described herein relate to neural recording and stimulation systems including tile-based organization, and methods of manufacturing the same.

### BACKGROUND

Technical advances have made whole-brain recording possible in small behaving animals (e.g., flies, zebrafish) using brain imaging microscopy such as two-photon microscopy, confocal microscopy, structured illumination, light-sheet microscopy. However, these approaches generate large data sets that are difficult to process, and therefore, these data sets may not be ideal for larger animals or humans and may not allow for high spatiotemporal recording. Additionally, microscopy techniques cannot achieve adequate tissue depth and resolution for human brain applications. Spatial and temporal resolution in brain recording can be important factors to achieve accurate and effective closed-loop stimulation. Electrical brain recording may improve spatial and temporal resolution, however, known approaches are not scalable to measure from large areas for closed-loop applications.

### SUMMARY

In some embodiments, an apparatus comprises a biocompatible substrate configured to be disposed on a surface of a brain, the biocompatible substrate including a plurality of nodes and a plurality of connectors interconnecting the plurality of nodes, the biocompatible substrate defining a plurality of openings therein, each of the plurality of nodes and the plurality of connectors including at least one encapsulation material. The plurality of nodes including: a first set of nodes, each node from the first set of nodes including (i) an associated set of electrodes from a plurality of electrodes and (ii) an associated analog circuit; a second set of nodes different than the first set of nodes, each node from the second set of nodes including a digital circuit; and a third set of nodes different than the first set of nodes and the second set of nodes, each node from the third set of nodes configured to supply power to the first set of nodes and to supply power to the second set of nodes.

In some embodiments, a method comprises coupling a biocompatible polymer substrate to a carrier substrate; applying a first encapsulation layer over a first surface of the biocompatible polymer substrate, to create a first intermediate structure; depositing a conductive metal layer on the first encapsulation layer and patterning the conductive metal layer to define one or more electrodes and one or more electrical traces, thereby producing a second intermediate structure that includes the one or more electrodes and the one or more electrical traces; applying a second encapsulation layer to at least a portion of the second intermediate structure; coupling a microelectronic device, from a set of at least one microelectronic devices, to each of the one or more electrical traces via the second encapsulation layer, thereby producing a third intermediate structure; applying a third encapsulation layer on at least a portion of the third intermediate structure, thereby producing a fourth intermediate structure; applying a layer of polymer material on at least portion of the fourth intermediate structure, thereby producing a fifth intermediate structure; and releasing the carrier substrate from the fifth intermediate structure to create an interconnect band.

In some embodiments, an apparatus comprises an interposer defining a plurality of openings therein, the interposer including: at least one electrode; a plurality of nodes, each node from the plurality of nodes including (i) an electrical contact disposed on a first surface of the interposer at that node and (ii) an electrical connection extending from the electrical contact to a second surface of the interposer opposite the first surface and at that node; and a first encapsulation material disposed around at least a portion of the interpose. The apparatus further comprising a plurality of integrated circuit chips coupled to the second surface of the interposer and electrically connected to the at least one electrode via the electrical connection, the integrated circuit chip including a second encapsulation material disposed on at least a portion thereof, the apparatus configured to be disposed on a surface of a brain of a user such that the at least one electrode contacts a surface of the brain of the user.

### BRIEF DESCRIPTION OF THE DRAWINGS

FIG. 1A is a schematic diagram of a neural device, according to some embodiments.

FIG. 1B is a schematic diagram of a neural device, according to some embodiments.

FIG. 2 is a schematic diagram of nodes of a neural device, according to some embodiments.

FIGS. 3A-3C are schematic diagrams of microelectronics of a first node of a first set of nodes, microelectronics of a second node of a second set of nodes, and microelectronics of a third node of a third set of nodes of a neural device, respectively, according to some embodiments.

FIG. 4A is a schematic diagram of an analog interconnect band of a neural device, according to some embodiments.

FIG. 4B is a schematic of a neural device including an interposer coupled to one or more integrated circuit chip, according to some embodiments.

FIG. 5A is a method of manufacturing an analog interconnect band of a neural device, according to some embodiments.

FIG. 5B is a method of manufacturing a neural device including an interposer coupled to one or more integrated circuit chips, according to some embodiments.

FIG. 6 is a neural device including a plurality of nodes connected by a plurality of interconnect bands, according to some embodiments.

FIG. 7A shows a first node, a second node, and a third node of a neural device, according to some embodiments.

FIGS. 7B-7C show the neural device including a plurality of nodes including the first node, the second node, and the third node of FIG. 7A connected by a plurality of interconnect bands, according to some embodiments.

FIG. 8A shows a first node, a second node, and a third node of a neural device, according to some embodiments.

FIGS. 8B-8C show the neural device including a plurality of nodes including the first node, the second node, and the third node of FIG. 8A connected by a plurality of interconnect bands, according to some embodiments.

FIGS. 9A-9P show a method of manufacturing an analog interconnect band of a neural device, according to some embodiments.

FIGS. 10A-10E show a method of manufacturing a neural device including an interposer coupled to an integrated circuit chip on a first side of the interposer, according to some embodiments.

FIG. 11 shows a cross-section of a neural device including an interposer including an integrated circuit chip coupled to a second side of the interposer, according to some embodiments.

FIG. 12 shows a top view of a neural device and a close-up/detail cross-sectional view of the neural device including an interposer coupled to integrated circuit chips on a first side and a second side of the interposer, according to some embodiments.

FIG. 13 is a schematic diagram of a neural device including one or more components organized in a grid configuration and a coil disposed around the one or more components organized in the grid configuration, according to some embodiments.

FIG. 14 is a schematic diagram of a neural device including a flexible circuit board coupled thereto, according to some embodiments.

FIG. 15 is a schematic diagram of a lace device coupled to a wire connector, according to some embodiments.

FIG. 16 is a schematic diagram of a neural device coupled to an external device including an inductive link, according to some embodiments.

FIG. 17 is a schematic of a node of a neural device including an integrated circuit chip coupled to a biocompatible substrate through electrical connectors extending through the integrated circuit chip, according to some embodiments.

### DETAILED DESCRIPTION

Whole-brain recording and/or recording of large areas across and/or within the brain can be a useful tool to understand how brain regions interact with one another during behavior. Whole-brain recording can also be useful for largescale neuromodulation applications. While advancements in imaging techniques such as two-photon microscopy, confocal microscopy, structured illumination, and light-sheep microscopy have enabled whole-brain imaging in small behaving animals (e.g., flies, zebrafish, rodents, etc.), whole-brain imaging (or imaging of large areas) presents challenges for larger animals and humans and/or moving subjects. These imaging approaches also generate large amounts of data (e.g., greater than 1 gigabit per second (GBPS)) that are heavy to process, do not allow for closed-loop stimulation, and lack spatiotemporal resolution. Spatial and temporal resolution in brain recording can be important factors to achieve accurate and effective closed-loop stimulation (e.g., stimulation in response to and/or based on recorded neural signals). Electrical brain recording may improve spatial and temporal resolution; however, known approaches for electrical brain recording face many challenges and are not ideal for large-scale applications. Large-scale applications as used herein can refer to implementing high channel counts across a large surface area coverage (e.g., channel counts greater than ≥˜1,000 over a surface area ≥25 mm×25 mm). Existing electrocorticography (ECOG) grids may cover large surface areas (e.g., up to 100 mm²), but suffer from low channel counts (e.g., only up to about 64 contacts). In contrast, the embodiments described herein can achieve large surface area coverage while maintaining high channel density.

Electrical whole-brain recording and stimulation systems may rely on a large number of electrodes to provide high spatial resolution. Combining high spatial resolution and high temporal resolution can greatly increase the amount of data for the system to transport, process, and analyze, and therefore, high speed digital lines may be desirable. However, high-speed digital lines can inject noise onto adjacent lines (e.g., analog lines) which can degrade the quality of recorded data (e.g., recorded analog neural signals). Neural signals can have a relatively low signal-to-noise ratio, and as such, amplifiers are often used to achieve a sufficient signal-to-noise ratio for subsequent signal processing. As used herein, an “input referred” noise can refer to a calculation to estimate a level of noise in the circuitry by dividing a total output noise by a total gain of the circuitry. Brain computer interface (BCI) recording electronics target an input referred noise on the order of μVᵣₘₛ, and therefore, low noise analog circuitry is desirable to enable signal quality that is sufficient enough to allow for proper neural pattern decoding. The injection of noise in analog signal lines caused by digital lines can significantly impact the ability to accurately process neural signals. Embodiments described herein address this challenge by spatially separating analog signal lines and digital signal lines to reduce or prevent crosstalk or interference therebetween. Embodiments described herein can achieve efficient data transmission, data processing, and/or processing/analysis of electrical signals while also maintaining a sufficient signal quality (e.g., signal-to-noise ratio, signal amplitude, and/or noise floor) of recorded neural signals to facilitate improved/high accuracy neural decoding. In some embodiments, the devices described herein may receive up to about 30 kilo-samples per second (kS/s) with 12 bit resolution per electrode channel (e.g., 360 kilobytes per second (KB/s)). In some embodiments, the microelectronics in the devices described herein (e.g., the ASICs described herein) may convert (e.g., analog to digital conversion, ADC) and compress analog data at the front end to reduce the amount of data transmitted along the digital signal lines such that a large number of channels can be supported. Devices described herein may achieve data transmission rates between about 300 MB/s and about 500 MB/s. In some embodiments, signal quality can be quantified based on the signal amplitude, the signal-to-noise ratio (SNR) (e.g., the signal amplitude divided by the noise amplitude), and/or a noise floor of the signals. In some embodiments, for an effective neural recording and/or stimulation system, it may be desirable for the SNR to be at least about 10 dB. It may be desirable to have a noise floor that is below an upper threshold of about 10 μV. In some embodiments, a noise floor below about 3 μV may be preferred. The devices described herein can achieve SNR of at least about 10 dB with a noise floor of at most about 10 μV.

Embodiments described herein include a neural recording and stimulation system (hereinafter, “neural device”) that includes a tile-based arrangement (e.g., lace, grid, lattice, mesh, etc.) to facilitate whole-brain recording (or recording from large brain areas) and/or stimulation with high spatiotemporal resolution. Embodiments described herein may be configured to measure local field potentials (LFP), electrocorticography (ECOG), microelectrocorticography (μECOG), and/or action potential recordings. Embodiments described herein may be configured to stimulate brain tissue of a subject using one or more electrode arrays. The neural devices described herein may be configured to record and/or stimulate the brain along its surface and/or at varying planes or depths beneath its surface. Embodiments described herein can facilitate brain stimulation in a closed-loop process by maintaining high signal quality while keeping data transmission rates sufficiently high.

Embodiments described herein can reduce noise caused by high-speed digital lines and therefore improve the quality of analog recordings. For example, embodiments described herein can physically separate high speed (and noisy) data lines from analog (low noise) data lines using a tile-based pattern. In some embodiments, analog and digital data lines can be physically separated by orienting the analog and digital lines along different axes, positioning the analog and digital data lines at/in/on different portions of the device, preventing physical overlap thereof, etc. A tile-based pattern can refer to a systematic arrangement of individual tiles (e.g., modules, elements, units, portions, etc.) to form a larger structure. Each of the individual tiles can be configured to function as a system that operates at a large scale while maintaining the quality of electrical signals from each individual tile. In some embodiments, the tiles can be organized in a pattern such that additional tiles can be configured to geometrically fit into the system. For example, the arrangement or orientation of the data lines may facilitate or accommodate the coupling of tiles to one another to form the larger structure. Embodiments described herein may include an auto-addressing feature for identifying each electrode in the system to organize data processing from a large number of inputs. For example, auto-addressing may allow neural devices described herein to track location of nodes and/or origin of signals. Neural devices described herein may further be configured to perform one or more node-level signal processing algorithms for closed-loop stimulation applications. The neural devices described herein may harness the efficiency and resolution of single node recording and provide a low-noise and scalable arrangement for whole-brain, closed-loop applications.

In some embodiments, the neural devices described herein may include a biocompatible material including a plurality of nodes and a plurality of connectors or bands (i.e., “interconnect bands”). In some embodiments, biocompatible material may refer to a material that minimizes or eliminates toxicity, immunogenicity, and/or any other adverse events when in contact with biological tissues. In some embodiments, biocompatible material may refer to a material that exhibits a tissue response profile that allows for chronic implantation at the intended sites. The biocompatible material may define a plurality of openings between the plurality of interconnect bands and the plurality of nodes. The plurality of nodes may include three sets of nodes with each set of nodes configured to perform a specific task. A first set of nodes may be configured for analog stimulation and recording of neural tissue, a second set of nodes may be configured for signal processing, and a third set of nodes may be configured for wireless power and/or data transmission. In some embodiments, the interconnect bands may physically and/or electrically connect nodes to one another. In some embodiments, the plurality of interconnect bands may include three sets of interconnect bands each configured to transmit a specific type of data. In some embodiments, a first set of interconnect bands may transmit analog data and connect analog nodes. The second set of interconnect bands may transmit digital data at a low or medium speed. The third set of interconnect bands may transmit digital data at a high speed. The digital interconnect bands and the analog interconnect bands may be physically separate and/or may not overlap to prevent contamination of the analog signal with noise, thereby increasing signal quality of analog recordings while maintaining data transmission speed.

The neural devices herein may include a tile-based structure, each tile including a grouping of nodes and interconnect bands. The tile-based structure may allow the neural device to be scalable to cover large areas while maintaining the signal quality and processing efficiency.

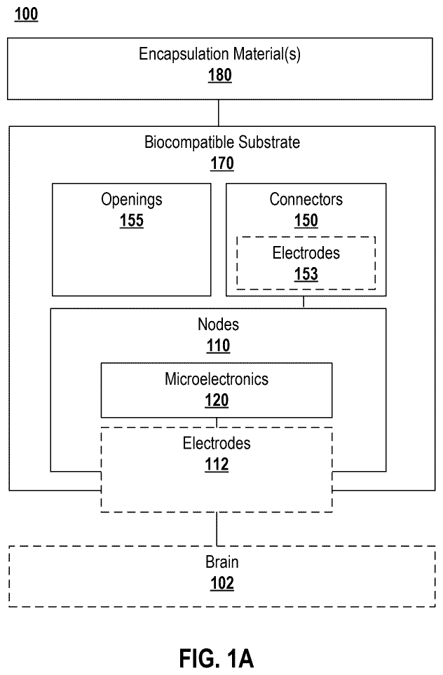

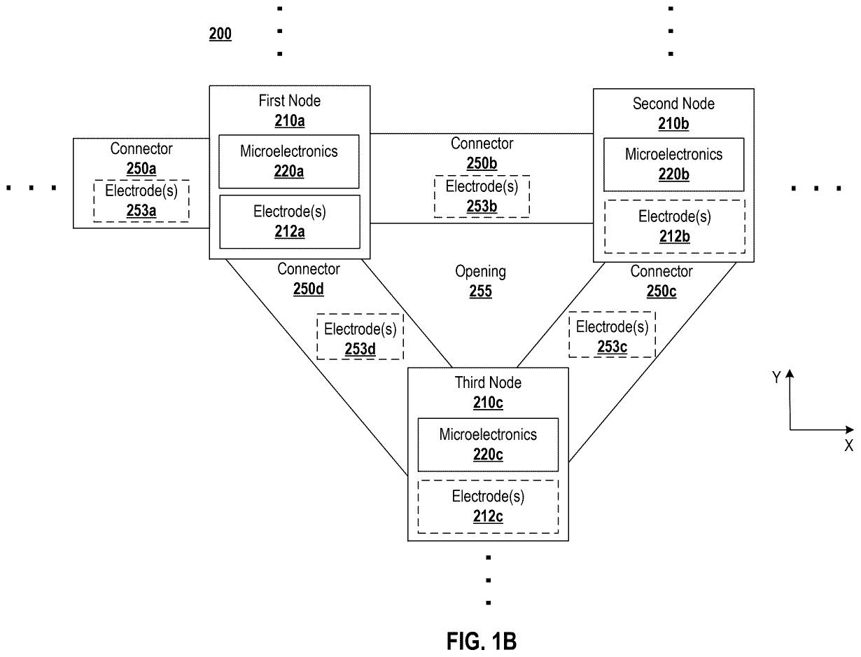

FIG. 1A is a schematic diagram of a neural device 100 configured to be disposed on a surface of a brain 102 of a patient, according to some embodiments. The neural device 100 may include a biocompatible substrate 170 including a plurality of nodes 110 and defining a plurality of openings 155 (e.g., recesses, gaps, apertures, holes, through-holes, cavities, voids, etc.). In some embodiments, the biocompatible substrate 170 may further include a plurality of connectors 150 (e.g., bands, strips, cables, cords, ribbons, wires, etc.) configured to physically and electrically connect the plurality of nodes 110. The connector(s) 150 may be referred to herein as interconnect bands. In some embodiments, the plurality of nodes 110 and the plurality of interconnect bands 150 may be formed from the same structure (e.g., the biocompatible substrate 170). In some embodiments, the interconnect bands 150 may be formed by removing portions of the biocompatible substrate 170 such that the biocompatible substrate 170 forms a mesh structure (e.g., lace, lattice, mesh, grid, tile structure, etc.). For example, the one or more openings 155 may be etched out of the biocompatible substrate 170 to form the plurality of nodes 110 and the plurality of interconnect bands 150. In some embodiments, the mesh structure may be defined by the plurality of interconnect bands 150 and the plurality of nodes 110. In some embodiments, the interconnect bands 150 may be manufactured separate from the plurality of nodes 110 and electrically connected (e.g., soldered) to the plurality of nodes 110.

In some embodiments, at least some of the plurality of nodes 110 can include microelectronics 120 including circuitry to operate the neural device 100. In some embodiments, each node from the plurality of nodes 110 can include associated or dedicated microelectronics 120. The microelectronic(s) 120 can include one or more circuits (e.g., analog circuits, digital circuits, communication interfaces, power supply, etc.). In some embodiments, the circuits of the microelectronics 120 can be flip-chip assembled onto the nodes 110 (e.g., onto the biocompatible substrate 170) and/or coupled to the nodes 110 via bump bonds. Alternatively or additionally, the neural device 100 may include microelectronics 120 for operation of the neural device 100 disposed adjacent to the mesh structure including the plurality of nodes 110 and the interconnect bands 150.

The neural device 100 may further include one or more electrodes 112, 153 coupled to the microelectronics 120 and configured to be disposed near the surface of the brain 102 and/or to extend into the surface of the brain 102. The one or more electrodes 112, 153 may be configured to record neural signals and/or stimulate neurons. In some embodiments, a first set of electrodes 112 may be coupled to each node from the plurality of nodes 110. In some embodiments, the first set of electrodes 112 may be disposed on a subset of nodes from the plurality of nodes 110. In some embodiments, the interconnect bands 150 may optionally include the second set of electrodes 153 disposed thereon. In some embodiments, the subset of nodes from the plurality of nodes 110 may each include a depth electrode array, and a subset of the interconnect bands 150 may each include one or more surface electrodes disposed thereon.

In some embodiments, each node from the plurality of nodes 110 may be disposed at an intersection point of a set of interconnect bands 150. The plurality of interconnect bands 150 may each include one or more conductive traces (e.g., data lines) running therethrough to electrically connect the plurality of nodes 110 and transmit data therebetween. In some embodiments, the plurality of interconnect bands 150 may include analog data lines, digital data lines, power lines, and/or communication lines. In some embodiments, the plurality of interconnect bands 150 may include interconnect bands having a first rate of data transmission, interconnect bands having a second rate of data transmission, and interconnect bands having a third rate of data transmission. For example, the plurality of interconnect bands can include low-speed or medium-speed data lines and/or high-speed data lines. The low-speed data lines may be suitable/configured to transmit analog signals due to lower noise of the low-speed data lines. The medium-speed and high-speed data lines may be capable of transmitting larger amounts of data more quickly than low-speed data lines, and therefore may be suitable for transmitting digital signals.

In some embodiments, the mesh structure (e.g., formed by the biocompatible substrate 170) may include a plurality of analog nodes coupled by interconnect bands 150 including analog data lines and at least one of digital data lines or power lines. Each of the analog nodes may include a depth electrode array and an associated integrated circuit. In some embodiments, the plurality of analog nodes may be coupled to a principal node configured to provide power to the plurality of analog nodes and/or communicate to external devices. In some embodiments, the principal node may be a node disposed at a terminal end of the mesh structure. Alternatively or additionally, the plurality of analog nodes can be coupled to a plurality of nodes configured to process signals, provide power, and/or communicate to external devices.

In some embodiments, the plurality of node(s) 110 can include at least the first subset of nodes and the second subset of nodes. The first subset of nodes and the second subset of nodes can be different from one another. For example, the first subset of nodes and the second subset of nodes can each be configured to perform a different function (and therefore can have one or more components that are different from, or that are not in common to, one another). In some embodiments, the first subset of nodes can be analog nodes configured to collect and/or transmit analog signals, and the second subset of nodes can be digital nodes configured to convert analog signals to digital signals, process analog and/or digital signals, and/or transmit digital signals. In some embodiments, the plurality of nodes can further include a third subset of nodes. In some embodiments, the third subset of nodes can be different (e.g., configured to perform a different function) than the first subset of nodes and/or the second subset of nodes. In some embodiments, the first subset of nodes, the second subset of nodes, and the third subset of nodes can be configured to communicate with one another such that the first subset of nodes, the second subset of nodes, and the third subset of nodes operate in coordination.

In some embodiments, the plurality of node(s) 110 may include at least one of: (i) a first subset of nodes (e.g., analog nodes) configured to control recording and stimulation of neural tissue, (ii) a second subset of nodes (e.g., digital nodes) configured to perform one or more signal processing operations, and/or (iii) a third node or third subset of nodes (e.g., communication nodes(s) or interface node(s)) configured to communicate with external devices and/or supply power to the first subset of nodes and the second subset of nodes. In some embodiments, the communication nodes may be configured to communicate information and/or power via radiofrequency (RF). In some embodiments, the plurality of interconnect bands 150 may include at least one of a first subset of bands, a second subset of bands, and a third subset of bands. In some embodiments, the first subset of bands may include the surface electrodes 153 (e.g., micro-electrocorticography μECOG) configured to contact a surface of the brain 102 to record brain activity. In some embodiments, the first subset of interconnect bands 150 may transmit analog data between the first subset of nodes. The second subset of interconnect bands 150 may include digital lines. In some embodiments, the second subset of interconnect bands 150 may include medium-speed digital lines. In some embodiments, the third subset of interconnect bands 130 may include high-speed digital lines.

In some embodiments, the neural device 100 can include the digital nodes “on-chip” with the plurality of analog nodes, meaning that the digital nodes can be distributed throughout a portion of the biocompatible substrate 170 including analog circuitry and configured to interface with the surface of the brain (e.g., the mesh structure). The digital nodes may not be disposed in a separate portion of the neural device 100 than the analog nodes. In some embodiments, the digital nodes may be configured to receive analog signals from nearby or neighboring analog nodes and perform one or more processes on the analog signals. In some embodiments, analog nodes can receive analog signals from neighboring interconnect bands 150 including electrodes. Therefore, analog signals may travel a shorter distance before being received by circuitry configured to process and/or communicate the analog signals. This organization of the plurality of nodes 110 can, for example, prevent signal degradation and improves efficiency in signal processing and transmission. In some embodiments, the analog nodes can be configured to convert analog signals to digital signals before communicating the signals to the digital nodes. In some embodiments, the analog nodes can communicate analog signals to the digital nodes. In some embodiments, the digital nodes may not include analog circuitry. In some embodiments, the analog nodes may not include digital circuitry. In some embodiments, the analog nodes may include analog circuitry and a digital interface. In some embodiments, the analog nodes may not include digital circuitry configured for signal processing of neural signals.

In some embodiments, the third subset of nodes can be or include communication nodes configured to supply power to the analog nodes and the digital nodes and/or to communicate signals received from the analog nodes and the digital nodes to an external device (e.g., a device external to the user). In some embodiments, the communication nodes may also be disposed “on-chip” with the plurality of analog nodes and/or the plurality of digital nodes. In some embodiments, the communication nodes may not be disposed in a separate portion of the neural devices 100 than the analog nodes. In some embodiments, the communication nodes can be configured to receive analog and/or digital signals from nearby or neighboring analog nodes and digital nodes and communicate the analog and/or digital signals to an external device. Therefore, analog signals and/or digital signals may travel a shorter distance before being received by the communication nodes configured to communicate the analog signals and/or digital signals to external devices, which improves signal quality and efficiency of signal transmission. In some embodiments, the digital nodes can convert the analog signals to digital signals, and the digital signals can be communicated to the communication nodes. The distribution of digital nodes and communication nodes can allow for a smaller amount of data to move through each node at one time, which allows the neural device 100 to process large amounts of data without overloading microelectronics 120. This also allows the neural device 100 to process large amounts of data without the use of large processors. In some embodiments, the analog nodes, the digital nodes, and the communication nodes can be organized in an alternating pattern such that neural device 100 includes sections (e.g., tiles as described herein), each of which can perform one or more of: analog functions (e.g., recording or stimulating), digital functions (e.g., signal processing), power harvesting, or communication functions).

Including analog and digital circuitry “on-chip” can lead to challenges around maintaining signal quality of analog signals due to noise caused by transmission of digital data. Devices that include digital and power circuitry separate from the analog circuitry (e.g., not “on-chip”) do not face these challenges. In some embodiments, the plurality of interconnect bands 150 that interconnect the plurality of nodes 110 of the neural device 100 can be configured for specialized functions. In some embodiments, the plurality of interconnect bands 150 can include at least a first subset or type of interconnect bands and a second subset or type of interconnect bands configured to perform different functions. In some embodiments, the first subset of interconnect bands can be configured to transmit analog signals or data and the second subset of interconnect bands can be configured to transmit digital signals or data. In some embodiments, the first subset of interconnect bands and the second subset of interconnect bands can be configured to connect the first subset of nodes and the second subset of nodes. In some embodiments, the plurality of interconnect bands 150 can include a third subset of interconnect bands. The third subset of interconnect bands 150 can be configured to transmit digital signals faster than the second subset of interconnect bands 150. In some embodiments, the third subset of interconnect bands 150 can be configured to electrically connect and/or transmit data between the digital nodes and the interface nodes. In some embodiments, each interconnect band from the plurality of interconnect bands 150 can be spatially separated from one another to prevent crosstalk between the interconnect bands. In some embodiments, the analog interconnect bands 150 and the digital interconnect bands 150 can be physically separated to prevent crosstalk therebetween. Therefore, the mesh structure of the biocompatible substrate 170 can include analog circuitry, digital circuitry, power circuitry, and communication circuitry without compromising the signal quality of analog signals collected.

In some embodiments, the first subset of interconnect bands 150 may be substantially physically separated from the second subset of interconnect bands 150 and/or the third subset of bands 150. For example, the first subset of interconnect bands 150 may not overlap with the second subset of interconnect bands 150 and/or the third subset of interconnect bands 150. In some embodiments, the first subset of interconnect bands 150 may be spaced at least a minimum distance from the second subset of interconnect bands 150 and/or the third set of interconnect bands 150. In some embodiments, the first subset of interconnect bands 150 may extend along a first and/or second axis, and the second subset of interconnect bands 150 may extend along a third axis and/or a fourth axis, the third axis and the fourth axis being different than the first axis and the second axis. Physical separation of the first subset of interconnect bands (e.g., the analog bands carrying analog signals) and the second and/or third subset of interconnect bands (e.g., the digital bands carrying digital signals) may prevent noise contamination from the second and/or third subset of interconnect bands into the first subset of interconnect bands. This may reduce noise on the first subset of interconnect bands and improve analog signal quality measured by the neural device 100.

In some embodiments, biocompatible substrate 170 may include a biocompatible polymer such as, for example, liquid crystal polymer (LCP), polyimide, silicon, or any suitable combination thereof. In some embodiments, the biocompatible substrate 170 may include an interposer (e.g., a silicon interposer), and the silicon interposer may be coupled (e.g., electrically connected) to an integrated circuit chip. In some embodiments, the silicon interposer may be coupled to an integrated circuit chip at each node 110 from the plurality of nodes 110. In some embodiments, the integrated circuit chip may be a logic die, and the silicon interposer may be electrically coupled to the logic die via through-silicon vias (TSV) and a bump bond. In some embodiments, the neural device 100 may include a biocompatible printed circuit board (PCB). The neural device 100 may be formed on a single wafer (e.g., silicon wafer) that is back-grinded to a predetermined thickness (e.g., between about 1 μm and 40 μm). In some embodiments, the predetermined thickness may be up to about 400 μm when the substrate includes LCP, for example. In some embodiments, the openings may be etched out of the back-grinded substrate between the interconnect bands 150.

In some embodiments, the neural device 100 may include one or more encapsulation materials 180 disposed on or around at least a portion of the neural device 100. In some embodiments, the encapsulation material(s) 180 may be disposed on the neural device 100 in one or more layers. In some embodiments, each of the plurality of nodes 110 and each of the plurality of interconnect bands 150 may be coated in one or more encapsulation materials 180. In some embodiments, all surfaces of the plurality of nodes 110 and the plurality of interconnect bands 150 may be coated in the encapsulation material(s) 180 aside from a portion of each electrode from the plurality of electrodes 112, 153. For example, a portion of each electrode 112, 153 may be exposed and configured to contact the brain 102 or be disposed near surface of the brain 102. In some embodiments, the encapsulation materials 180 may be disposed around the biocompatible substrate 170 during fabrication and before singulation such that the encapsulation material(s) 180 covers all sides of the plurality of node(s) 110 and the interconnect bands 150. In some embodiments, the encapsulation material 180 may include at least one of silicon carbide (SiC), doped SiC, polyimide, parylene-C, liquid crystal polymer (LCP), alumina, silicon dioxide (SiO₂), Polyurethane (PU), (medical grade) Silicone or any suitable combination thereof. In some embodiments, the SiC may include amorphous SiC and/or polycrystalline SiC. The encapsulation material(s) 180 may prevent degradation of the neural device 100 due to exposure to biological materials. In some embodiments, the encapsulation materials 180 may increase longevity of the neural device 100.

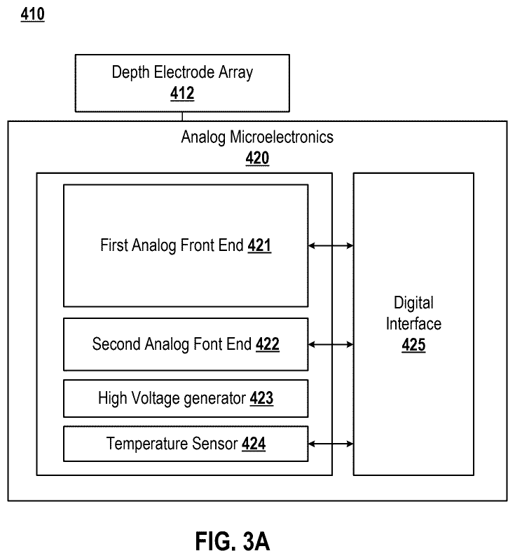

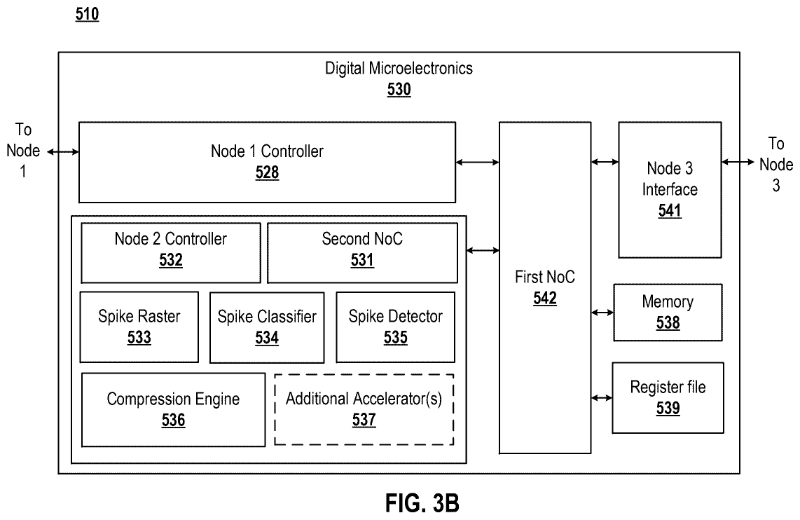

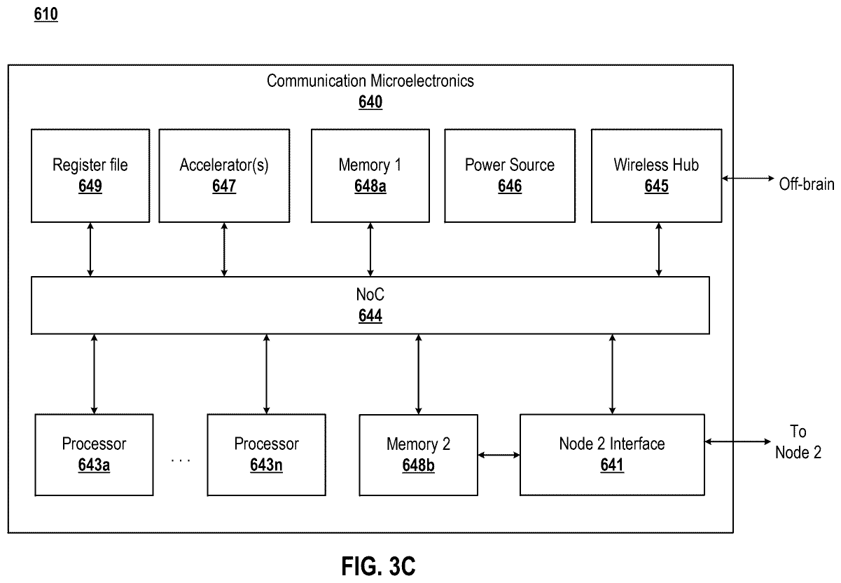

In some embodiments, the neural device 100 may be formed of a plurality of tiles, each tile including a set of nodes 110 and associated interconnect bands 150. For example, one or more nodes from the first set of nodes, one or more nodes from the second set of nodes, and/or one or more nodes from the third set of nodes may be coupled together to form a tile. The tiles may be coupled together to form the mesh structure. The tiles may facilitate the neural device 100 to be scalable. For example, an area covered by the neural device 100 may be expanded by including additional tiles. In some implementations, each tile may include the first subset of nodes, the second subset of nodes, and the third subset of nodes such that each tile is configured to perform all of the functions of each subset of nodes. Each of the nodes in a given tile may be configured to work in coordination such that each tile can function to record, analyze, and communicate signals independently of the other tiles. Alternatively, the tiles of the neural device 100 may operate interdependently and signals may be communicated between tiles. For example, signals collected from one portion of the neural device 100 can be used to control a different portion of the neural device 100 for stimulation. In some embodiments, neural signals collected across a plurality of tiles can be processed separately or can be pooled and processed together, depending on the application. In some embodiments, auto-addressing can be used to track operation of tiles across the neural device (described further below, with reference to FIGS. 3A-3C).

In some embodiments, each node from the plurality of nodes 110 may include any suitable shape such as a circle, a square, a rectangle, a hexagon, an octagon, or a combination thereof. In some embodiments, the plurality of nodes 110 may include a shape having 3 sides, 4 sides, 5 sides, 6 sides, 8 sides, etc. In some embodiments, an interconnect band 150 may extend from each side of a respective node 110. In some embodiments, the mesh structure may have any suitable configuration such as a square configuration, a hexagonal configuration, an octagonal configuration, etc. For example, each tile may include a square shape in which the interconnect bands 150 of a tile define a square shape, and the nodes of the tile are disposed at the vertices of the square shape. In some embodiments, the tile may include a hexagonal shape in which the interconnect bands 150 of the node define a hexagon, with the nodes of the tile are disposed at the vertices of the hexagon. In some embodiments, the tiles may form a hexagonal shape such that the mesh structure forms a honeycomb pattern.

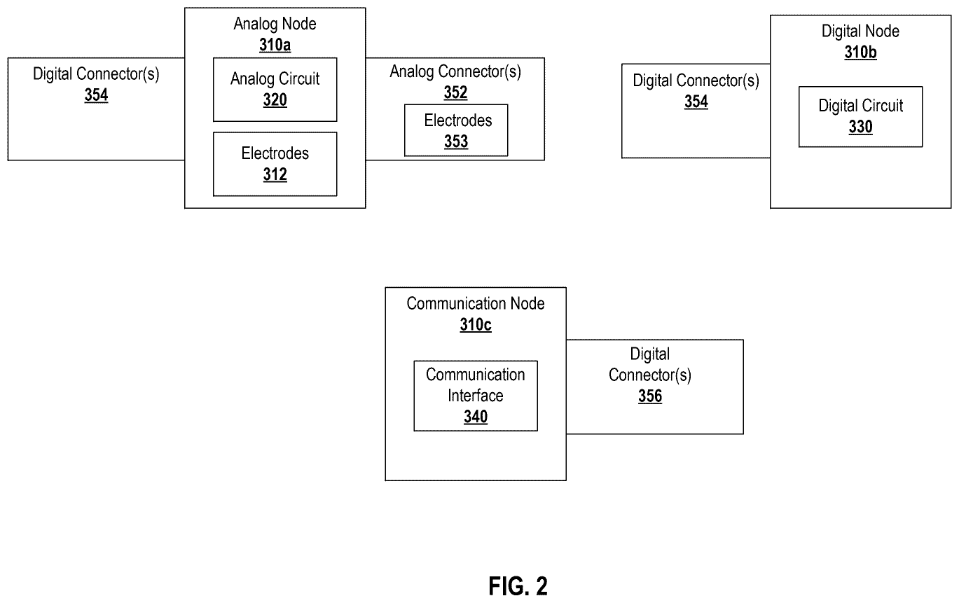

The neural device 100 may include any suitable number of nodes 110. In some embodiments, the neural device 100 may include between about 3 nodes and about 1024 nodes, inclusive of any ranges and subranges therebetween. The neural device 100 may include any suitable number of interconnect bands 150 connecting the nodes 110. In some embodiments, the neural device 100 may include between about 3 interconnect bands and about 8192 interconnect bands, inclusive of all ranges and subranges therebetween. Further details of the plurality of nodes and the plurality of bands are described in further detail with respect to FIGS. 2-4C. In some embodiments, the neural device 100 may be configured to cover a surface area between about 5 mm² and about 650 mm², inclusive of all ranges and subranges therebetween. In some embodiments, the neural device 100 may be configured to cover a surface area between about 10 mm² and about 100 mm², inclusive of all ranges and subranges therebetween. In some embodiments, the neural device 100 may be configured to cover a surface area up to about 450 cm², inclusive of all ranges and subranges therebetween. In some embodiments, the neural device 100 may be configured to cover a surface area up to about 100 cm², inclusive of all ranges and subranges therebetween.

In some embodiments, the microelectronics 120 of the plurality of nodes 110 may include integrated circuit (IC) chips (e.g., an application specific integrated circuit (ASIC)), each IC chip from the plurality of IC chips being sufficiently rigid to prevent anisotropic electronic property changes during operation thereof. In some embodiments, each node from the plurality of nodes 110 may be sufficiently rigid to prevent anisotropic electronic property changes during operation thereof. In some embodiments, each interconnect band from the plurality of interconnect bands 150 may be flexible such that the neural device 110, when disposed on a brain surface 102 of a patient, fits to (e.g., flexes with, accommodates, bends to, etc.) a curvature of the brain surface 102. Therefore, the interconnect bands 150 are configured to be sufficiently flexible to allow the device 100 to flex to the curvature of the brain 102 while the plurality of nodes 110 are configured to be sufficiently rigid to prevent anisotropic electronic property changes when the neural device 100 curves to the surface of the brain 102. In some embodiments, an area of each of the plurality of nodes 110 may be smaller than a predetermined threshold such that the plurality of nodes 110 do not prevent flexing of the plurality of interconnect bands. For example, each node from the plurality of nodes 110 may have an area less than about 0.5 mm², less than about 0.75 mm², less than about 1 mm², less than about 1.25 mm², less than about 1.5 mm², less than about 1.75 mm², less than about 2 mm². In some embodiments, a total thickness of the neural device 110 may be in a range of about 1 μm to about 1000 μm, inclusive of all ranges and subranges therebetween. In some embodiments, a total thickness of the neural device 110 may be in a range of about 5 μm to about 30 μm, inclusive of all ranges and subranges therebetween.

FIG. 1B is a schematic diagram of a neural device 200 including at least a first node 210a, a second node 210b, and a third node 210c connected by a plurality of connectors (e.g., interconnect bands) 250a, 250b, 250c, 250d. As shown, the nodes 210a, 210b, 210c and the interconnect bands 250a, 250b, 250c, 250d may define an openings 255 therebetween. In some embodiments, the nodes 210a, 210b, 210c may be coupled to additional nodes to form a large mesh or lattice structure. The neural device 200 is shown along an x-y axis, with the x-y plane corresponding to a surface of the brain.

In some embodiments, the first node 210a, the second node 210b, and the third node 210c may all have the same structure and function. For example, each of the nodes 210a, 210b, 210c may include one or more electrodes 212a and associated microelectronics 220a. In some embodiments, the nodes 210a, 210b, 210c may be configured to record analog signal from neural tissue and/or stimulate the neural tissue via the electrode(s) 212a. The microelectronics 220a, 220b, 220c may include associated analog front-end circuitry for recording and/or stimulating neural tissue and/or digital circuitry for performing one or more signal processing algorithms. In some embodiments, the electrode(s) 212a, 212b, 212c may be a first set of electrodes, and the interconnect bands 250a, 250b, 250c, 250d may optionally include a second set of electrode(s) 253a, 253b, 253c, 253d. The first set of electrode(s) 212a, 212b, 212c may include depth electrodes. For example, the first set of electrode(s) 212a, 212b, 212c may include a depth electrode array. The second set of electrodes 253a, 253b, 253c, 253d may include one or more surface electrode (μECOG). The interconnect band(s) 250a, 250b, 250c, 250d may be configured to transmit analog data, digital data, and/or power therethrough. For example, the interconnect band(s) 250a, 250b, 250c, 250d may be configured to transport analog information such as neural signal recordings towards the nodes 210a, 210b, 210c and/or stimulation waveforms from the nodes 210a, 210b, 210c towards the electrodes 253a, 253b, 253c, 253d). In some embodiments, the nodes 210a, 210b, 210c may be coupled to a plurality of additional nodes to form a lattice or mesh structure. In some embodiments, the nodes 210a, 210b, 210c may be coupled to a principal node (not shown) configured to provide power and/or wireless communication to the plurality of nodes 210a, 210b, 210c. The principal node may be disposed at a terminal end (e.g., a bottom edge) of the neural device 200.

Alternatively, the nodes 210a, 210b, 210c may include different structures and/or perform different functions. For example, the first node 210a may be configured as a first analog node and the second node 210b may be configured as a second analog node. The first analog node and the second analog node may be functionally similar or the same, but the geometry of the interconnect bands 250a, 250b, 250c, 250d extending therefrom may differ. For example, the first analog node and the second analog node may have a mirrored structure to facilitate the formation of the mesh or lattice structure. For example, a shape formed by the interconnect bands 250a, 250b, 250c, 250d extending from the first node 210a may be different than a shape formed by the interconnect bands 250a, 250b, 250c, 250d extending from the second node 210b. The first analog node and the second analog node may each have one or more analog interconnect bands extending therefrom to transmit analog information therebetween. The first analog node and the second analog may each include one or more digital interconnect bands extending therefrom to transmit digital information. The third node 210c may be configured as a digital node. The digital node may not include electrode(s) 212c. In such embodiments, the microelectronics 220c of the third node 250 may be configured to perform node-level signal processing operations. The digital node may include a digital interconnect band 250c extending therefrom to transmit digital signals. The digital interconnect band 250c may not include surface electrodes 232c.

Alternatively, the nodes 210a, 210b, 210c may all include different structures and/or perform different functions. For example, the first node 210a may be configured as an analog node, the second node 210b may be configured as a communication node, and the third node 210c may be configured as the digital node. In some embodiments, the interconnect bands 250a, 250b, 250c, 250d may include analog connectors, medium speed digital connectors, and high-speed digital connectors. The function of the analog, digital, and communication nodes are described in further in FIGS. 2-3C.

FIG. 2 is a schematic diagram of a plurality of nodes 310a, 310b, 310c of a neural device, according to some embodiments. In some embodiments, the first set of nodes 310a (e.g., analog nodes 310a), the second set of nodes 310b (e.g., digital nodes 310b), and the third set of nodes 310c (e.g., communication nodes 310c) may be coupled together by a plurality of connectors 352, 354, 356 (e.g., bands, interconnect bands, strips, cables, cords, ribbons, wires, etc.). In some embodiments, the plurality of interconnect bands 352, 353, 354 may include a first subset of interconnect bands (e.g., analog connector(s) 352), a second subset of interconnect bands (e.g., digital connector(s) 354), and a third subset of bands (e.g., digital connector(s) 356).

In some embodiments, the analog node(s) 310a may include (1) an associated set of electrodes 312 from a first plurality of electrodes and (2) an associated analog circuit 320. In some embodiments, the analog node(s) 310a may being interconnected via the analog connector(s) 352, the analog connector(s) 352 may include a second plurality of electrodes 353. In some embodiments, the first set of electrodes 312 may include an array of depth electrodes, and the second set of electrodes 353 may include a plurality of surface electrodes (e.g., μECOG). In some embodiments, the first set of electrodes 312 can include surface electrodes. In some embodiments, the analog node(s) 310 is configured to perform at least one of recording neural data or stimulating a region of a brain via at least one of the array of depth electrodes 312 or the plurality of surface electrodes 353. The analog circuit 320 may include any suitable circuitry for recording analog brain signals and/or stimulation via the depth electrodes and/or the surface electrodes. For example, the analog circuit 320 can include one or more amplifiers, filters, etc. In some embodiments, the analog node 310a may include a digital circuit.

The digital node(s) 310b may include a digital circuit 330 (e.g., one or more IC's). In some embodiments, the digital node(s) 310b may include a digital circuit 330 and/or an analog circuit. In some embodiments, the digital circuit 330 may include any suitable circuitry for signal processing (e.g., signal processing at the node level). For example, the digital circuit 330 may be configured to execute a signal processing algorithm for at least one of spike detection, threshold estimation, data compression, and/or early-stage processing of neural signals recorded by the analog node(s) 310a. In some embodiments, the digital circuit 330 may include a system-on-chip (SoC). In some embodiments, the SoC may include a plurality of processors. In some embodiments, the SoC may include at least one of energy-efficiency network-on-chip (NoC), dedicated energy-efficient hardware accelerators configured to perform complex signal processing algorithms (e.g., more complex processing than the analog circuit 320). In some embodiments, the one or more processors may be configured to perform at least one of training, spike classification, or determination of spike timing for closed-loop autonomous applications. In some embodiments, the circuitry for recording and stimulating neurons (e.g., analog circuitry) may be disposed on separate nodes than the circuitry configured for signal processing (e.g., spike detection, etc.) and communication (e.g., of power or data).

The communication node(s) 310c may be configured to supply power to the first set of nodes 310a and to supply power to the second set of nodes 310b. In some embodiments, the communication node(s) 310c may be configured to communicate (e.g., wirelessly) with external devices. For example, the communication node(s) 310c may include a communication interface 340 configured to send data collected by the neural device to the external device. In some embodiments, the communication node(s) 310c may further be configured to obtain power from an external source (e.g., through wireless charging) and supply the power to the neural device.

In some embodiments, the analog connectors 352 may be configured to transport analog information (e.g., neural signal recordings towards the nodes and/or stimulation waveforms from the nodes towards the electrodes). In some embodiments, the analog connectors 352 may extend between the analog node(s) 310a. In some embodiments, the analog connectors 352 may only be connected to analog node(s) 310a. In some embodiments, the digital connectors 354 may be configured to transport digital information between the nodes 310a, 310b, 310c. The digital connectors 354 may be coupled to the analog node(s) 310a, the digital node(s) 310b, and/or the communication node(s) 310c. In some embodiments, the digital connectors 354 may be configured as low or medium speed digital lines. In some embodiments, the digital connectors 356 may be configured as high-speed digital lines configured to communicate power to each of the nodes (e.g., the first set of nodes 310a and the second set of nodes 310b).

In some embodiments, the first subset of bands (e.g., the analog connectors 352) may extend along a first axis and a second axis, and the second and/or third subset of bands may extend along a third axis and a fourth axis, the third axis and the fourth axis different than the first axis and the second axis such that the second subset of nodes and the third subset of nodes do not physical overlap with one another (or a physically separate from one another).

In some embodiments, the neural device may be configured to auto-address portions of the neural device to keep track of the scalable architecture and the higher number of channels. Scaling to a high number of channels (e.g., up to millions) can lead to two challenges including: high power consumption and keeping track of the recorded channel. The high data throughput is the main source of power consumption, which can be mitigated by a compression engine in the digital circuit 330 of the second node 310b (described further in FIG. 3B). Keeping track of the physical location of the recorded channels requires auto-addressing of the individual electrodes, the individual integrated circuits, and the individual tiles.

The analog nodes 310a may be configured to address the electrodes 312, 353. For example, the analog nodes 310a can include microelectronics configured to track which electrode(s) of the electrodes 312, 353 are recording or stimulating at a given time or time window. The digital nodes 310b may be configured to address the analog nodes 310a within a tile. For example, the digital nodes 310b can include microelectronics configured to track which node of the analog nodes 310a are receiving or sending signals at a given time or time window. The communication nodes 310c may be configured to address the tiles (e.g., groupings of nodes and interconnect bands) within the large neural device structure. For example, the communication nodes 310c can be configured to identify which tile the communication node 310c is located in and send a signal corresponding to the identity of the tile to the external device along with any data the communication node 310c communicates. Each electrode 312, 353 may correspond to a unique identification number defined by the electrode position, the circuit ID, and the tile ID, which can determine (e.g., automatically) at startup during the power-on sequence. The auto-addressing sequence may include a combination of hardware-based auto-addressing (e.g., a finite state machine) on the analog circuit 320 and/or the digital circuit, and a software-based auto-addressing using a processor (e.g., one or more computer processing unit (CPU)) to support an infinite number of channels.

FIGS. 3A-3C are schematic diagrams of microelectronics of each of a first node 410 of a first set of nodes, a second node 510 of a second set of nodes, and a third node 610 of a third set of nodes of a neural device, respectively, according to some embodiments. The first node 410 may be configured as an analog node including analog microelectronics 420. As shown in FIG. 3A, the first node 410 may include a depth electrode array 412 (e.g., a UTAH array) coupled thereto and one or more analog circuits 421, 422 (e.g., one or more analog ASICs). In some embodiments, the depth electrode array 412 may include a 9 electrode by 4 electrode array (e.g., a total of 36 electrodes). In some embodiments, 4 of the 32 electrodes may be reference electrodes.

The analog circuits may include a first analog front end 421 operatively coupled to the depth electrode array 412, and a second analog front end 422 operatively coupled to one or more surface electrodes (e.g., surface electrodes 253). In some embodiments, the second analog front end 422 may be configured to stimulate at lower frequencies with higher current than the first analog front end 421. High frequency stimulation and lower current may be effective when the stimulating electrodes are in closer proximity to a small number of neurons (e.g., 1 to about 10 neurons at a time), whereas low frequency stimulation with high current may be more effective when stimulating larger populations of neurons (e.g., 10 neurons to greater than 1000 neurons) from a further distance. Because the first analog front end 421 is coupled to the depth electrode array 412, which interacts with neurons on a smaller scale and is in closer proximity to the neurons, the first analog front end 421 can be configured to apply high frequency stimulation to effectively stimulate surrounding neurons. The second analog front end 422 is coupled to the surface electrodes, which are a further distance from the tissue of interest and interact with a large number of neurons and therefore, the second analog front end 422 can be configured to apply lower frequency stimulation to effectively stimulate. In some embodiments, the first node 410 may include a digital interface (e.g., high speed digital interface) 425 configured to (1) increase speed of data transmission and/or processing, (2) to perform auto-addressing, and (3) for performing closed-loop functions. In some embodiments, the microelectronics of the analog node 410 can be configured for less complex processing and consume lower power than the digital nodes or communication nodes. The lower complexity of the microelectronics on the analog nodes 410 allows the neural device to include a higher number of analog nodes 410 without draining power. In some embodiments, the first node 410 may include a high voltage generator 423. In some embodiments, the high voltage generator 423 may include an on-board charge-pump to generate high voltages. In some embodiments, the high voltage generator 423 can be configured to supply power for the stimulating circuitry. For example, the high voltage generator 423 can be configured to power amplifiers in the analog circuitry. In some embodiments, the amplifiers in the analog circuitry may be configured to amplify output of signals received from digital-to-analog converters from the digital nodes such that the analog nodes can output signals having a voltage above a stimulation threshold. Because the main voltage rail of the ASIC(s) of the analog node may be too low for some stimulation conditions, the charge pump can be used to reach higher voltages for stimulation. In some embodiments, the first node 410 may include one or more sensors including a temperature sensor 242 to prevent overheating of the device (e.g., the electrodes) and promote patient safety.

FIG. 3B shows a second node (e.g., the digital node) 510. The digital node 510 may include digital microelectronics 530 configured to perform on-chip data processing, which can be important for accommodating a high number of channels while reducing off-chip data rate. This facilitates real-time or near real-time applications, closed-loop studies, and medical device applications. The digital node 510 may be coupled to the analog node 410 via a first controller (e.g., “Node 1 Controller 528”). The Node 1 controller 528 can allow communication between analog node 410 and the data recording and stimulation circuitries of the digital node 510, respectively. In some embodiments, the Node 1 controller 528 may include packet-based protocol generated inside the controller to facilitate detailed debugging of the digital node 532.

The second node 510 can further a second controller 532 (e.g., “Node 2 Controller) configured to control operation of the components of the digital microelectronics 530. In some embodiments, the second controller 532 may include one or more processors and/or accelerators configured to execute one or more functions or algorithms (e.g., algorithms for processing neural signals and/or generating stimulation waveforms). For example, the second node 510 may include processors and/or accelerators including at least one of: (1) a spike detector (SD) 535, (2) a spike classifier (SC) 534, (3) a spike raster 533, a compression engine 536, and/or (4) one or more other additional accelerators 537. In some embodiments, the one or more functions or algorithms can include, for example, recurrent neural networks, transformer networks, and/or convolutional networks (e.g., 1D or 2D CNN). In some embodiments, the one or more functions or algorithms can include, for example, machine learning algorithms trained using both supervised and unsupervised learning. In some embodiments, the one or more functions or algorithms can include linear discriminators and/or threshold detection algorithms.

The spike detector 535 may be configured to extract action potentials (i.e., neuronal spikes) from background noise and only transmit detected spikes (e.g., to the communication nodes described herein), thereby reducing a total amount of data transmitted. The SD 535 can reduce transmission power by about 70-90% for a 10,000-channel system. The SD 535 can also reduce the data rate by about 90-99% compared to raw data transmission.

The spike classifier (i.e., spike sorting algorithm) 534 may include an artificial-intelligence (AI)-supporting feature that enables each node to autonomously recognize the sources of detected spikes. For example, the spike classifier (SC) 534 may determine an electrode, a node, and/or a tile from which a signal or set of signals is recorded. The AI feature of the SC 534 may be configured to decode activity from the brain (e.g., decoding neural activity corresponding to a task or a perception) and output corresponding information. SC 534 can further reduce the transmission power by 90-99% and the data rate by 90-100%. Moreover, the neural decoding algorithms can leverage the SC 534 to perform real-time and low-power closed-loop applications.

Spike raster (SR) 533 can determine the spike time interval (e.g., an amount of time between neural spiking) to analyze the neurons' behavior (e.g., spike rate, neural encoding, etc.) in response to various stimuli from outside world. With SR 533, neural encoding can be performed in real-time and in a power-efficient mode.

The compression engine 536 may be configured to perform single- and/or multi-compression 536 of data. The digital microelectronics 530 may include a first network-on-a-chip (NoC) 542 and a second NoC 531 configured to interface to accelerators 533, 534, 535, 536, 537. In some embodiments, the first NoC 542 and the second NoC 531 can be specialized for different tasks. In some embodiments, the second NoC 531 may be hardware-efficient and/or lightweight. The second NoC 531 may be low-speed. The second NoC 531 may handle data routing between different signal processing blocks (e.g., in any order needed). The first NoC 542 may be configured to handle high-speed data routing between memory 538, register file 539, and/or the other nodes (e.g., Node 1 and Node 3) at low power. Although not shown, additional acceleration engines 537 can be added to the digital node 510 such as, for example, local static random access (SRAM), adaptive threshold estimator (ATE), embedded field-programmable gate array (eFPGA), temperature sensor (TS), etc. As shown, the digital node 510 may be coupled to the communication node (e.g., communication node 610 shown in FIG. 3C) via the Node 3 interface 541. The interface 541 may include an asynchronous (ASYNC) interface 541.

Synchronizing all digital circuits via a clock signal may not be feasible in a device that covers a large brain area because clock signals traveling long distances may experience delay. Therefore, embodiments described herein may implement a hybrid clocking approach in which nodes nearby to one another may be synchronized, while nodes having a distance therebetween above a threshold may be asynchronous. In some embodiments, the Node 3 interface 541 may allow the digital node 510 to be a locally synchronous node (e.g., synchronous to nodes nearby) but a globally asynchronous cluster, which reduces global clocking distribution concerns in a network and improves scalability.

In some embodiments, the digital microelectronics 530 may include a status identifier (e.g., “register file 539”) configured to provide static and dynamic configurations of each element or component in the digital node 510, as well as the ability to monitor the status of each element.

The digital microelectronics 530 may further include a memory 538 configured to store raw data and/or at least partially processed data. In some embodiments, the memory 538 may be configured to store code for one or more instructions, algorithms, or processes executed by the digital node 510. For example, the memory 538 may store instructions to perform spike detection 535, spike classifier 534, spike raster 533, or other functions of accelerators 537. In some embodiments, the memory 538 may store neural signals and/or waveforms for stimulation. In some embodiments, the memory 538 may store information related to auto-addressing to track channel location and signal origins during operation of the neural device. The memory 538 may communicate code for instructions, algorithms, or processes to the processors and/or accelerators of the second node 510 via the first NoC 542 and second NoC 531. In some embodiments, the digital microelectronics 530 can further include a digital-to-analog converter such that the digital microelectronics 530 output analog signals (e.g., corresponding to stimulation waveforms) to respective analog nodes.

FIG. 3C shows a third node (e.g., communication node) 610. The communication node 610 may be configured to communicate with external (e.g., off-brain) components. In some embodiments, the communication node 610 may communicate with external components via radiofrequency (RF). In some embodiments, the communication node 610 may supply power to all nodes on the neural device and/or integrate additional on-chip data processing shared among adjacent digital nodes (e.g., digital node 510). The wireless hub 645 may be configured to communicate with off-brain devices. The power source 646 may provide stable power supplies for the on-the-network components of the communication node 610.

In some embodiments, the communication node 610 microelectronics 640 may include one or more processor(s) 643a. 643n. The processor 643a-643n may include any suitable processor. In some embodiments, the processor 643a-643n may include computer processing units (CPU). The microelectronics 640 may further include one or more memories. For example, the microelectronics 640 may include a first memory 648a (e.g., MEM L1) and a second memory 648b (e.g., MEM L2). In some embodiments, the processors 643a-643n and the memories 648a, 648b may provide flexibility and self-trainability features. Each processor 643a-643n may include a power-off capability to drastically reduce leakage power while the processors 643a-643n are not operating (e.g., while they are in sleep mode). The memories 648a, 648b may be shared among all the processors 634a-643n and may store instructions and/or data (e.g., raw and/or processed neural signals, waveforms, auto-addressing data, etc.) The accelerator(s) 647 may be configured to offload the parallel computation from the processors 643a-643n to reduce dynamic power (e.g., general multiplier-accumulator unit for vector computation). In some embodiments, the register file 649 may provide static and dynamic configurations of every element in the communication node 610, as well as the ability to monitor the status of each element. The Network-on-a-chip (NoC) 644 may communicate information within the communication node 610. For example, the NoC 644 may communicate information and/or data between components of the communication node 610. In some embodiments, the communication node 610 may include an interface (e.g., Node 2 interface) to couple the communication node 610 to a respective digital node or set of digital nodes (e.g., digital node 510). In some embodiments, the digital nodes (e.g., digital node 510) can act as a bridge or interface configured to communicate signals between the analog nodes (e.g., analog node 410) and the communication node (e.g., communication node 610).

The one or more processors of the analog node 410, the digital node 510, and/or the communication node 610 can be any suitable processing device(s) configured to run and/or execute a set of instructions or code. For example, the processors of the analog node 410, the digital node 510, and/or the communication node 610 may include one or more data processors, physics processing units, digital signal processors (DSP), analog signal processors, mixed-signal processors, machine learning processors, deep learning processors, finite state machines (FSM), compression processors (e.g., data compression to reduce data rate and/or memory requirements), encryption processors (e.g., for secure wireless data and/or power transfer), and/or the like. The processors can be, for example, a general-purpose processor, central processing unit (CPU), microprocessor, microcontroller, Field Programmable Gate Array (FPGA), an Application Specific Integrated Circuit (ASIC), a processor board, a virtual processor, and/or the like. In some embodiments, computations are shared between a field-programmable gate array (FPGA) chip and a microcontroller to optimize for real-time or near real-time operations. The processors can be configured to run and/or execute application processes and/or other modules, processes and/or functions associated with the system. The underlying device technologies may be provided in a variety of component types, for example, metal-oxide semiconductor field-effect transistor (MOSFET) technologies like complementary metal-oxide semiconductor (CMOS), bipolar technologies like generative adversarial network (GAN), polymer technologies (e.g., silicon-conjugated polymer and metal-conjugated polymer-metal structures), mixed analog and digital, and/or the like.

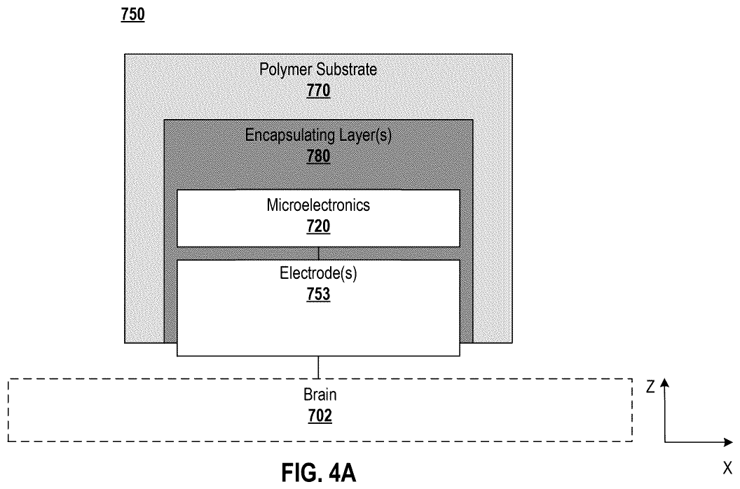

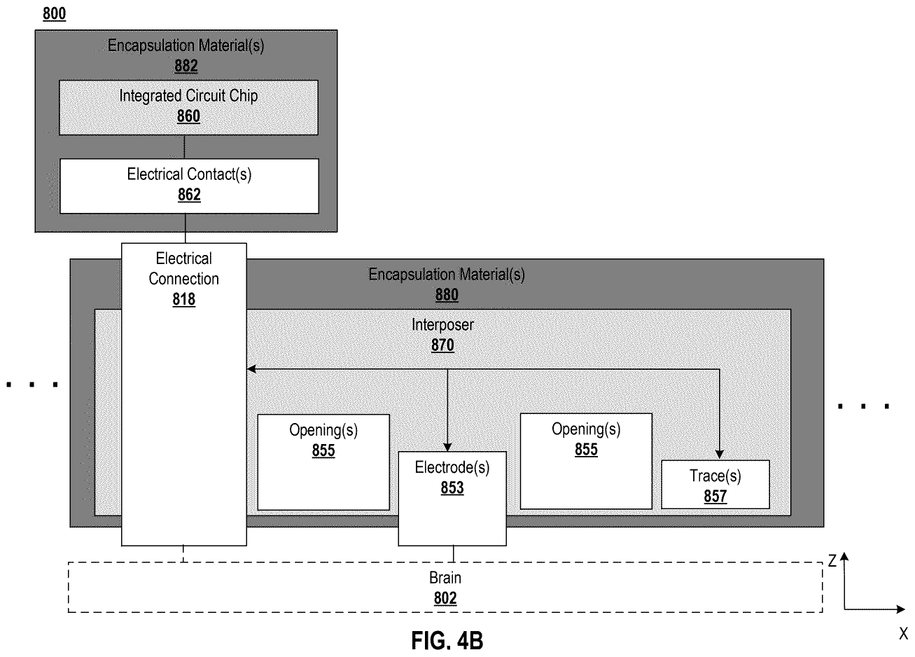

FIG. 4A is a schematic diagram of an interconnect band 750 (e.g., an analog interconnect band) of a neural device, according to some embodiments. The interconnect band 750 is shown on an x-z axis where the x-axis extends substantially along the surface of the brain 702 and the z-axis extends substantially perpendicular to the surface of the brain 702. As shown, the interconnect band 750 may include one or more electrodes 753 and associated microelectronics 720. The one or more electrodes 753 may be surface electrodes disposed on a surface of the interconnect band 750 configured to face towards the brain 702 of the user. The microelectronics 720 may include any suitable circuitry to transport and/or partially process and/or analyze biosignals (e.g., action potentials, LFPs, etc.) measured by the electrode(s) 753. In some embodiments, the microelectronics 720 may include silicon with an integrated circuit. In some embodiments, the microelectronics 720 can refer to microelectronics 720 at the nodes between the plurality of interconnect bands 750. In some embodiments, the microelectronics 702 may be coupled to the electrodes 753 via a bump bond. The microelectronics 720 and the electrode(s) 753 may be coated in one or more encapsulating layer(s) 780. In some embodiments, an entirety of the microelectronics 720 and electrode(s) 753 may be covered in the encapsulation material aside from a portion of each electrode 753 configured to contact the brain 702. In some embodiments, the microelectronics 720 and the electrode(s) 753 may be coupled to a polymer substrate 770. In some embodiments, the polymer substrate 770 may be disposed over or around the encapsulating layer(s) 780. In some embodiments, the polymer substrate 770 may be disposed over an entirety of the interconnect band 750 aside from a portion of each electrode 753 configured to contact the brain 702.

In some embodiments, the polymer substrate 770 may include at least one of polyimide and/or LCP. In some embodiments, the encapsulating layer(s) 780 may include a material such as, for example, SiC and/or parylene C. The electrodes 753 may include any suitable conductive material such as, for example, tantalum, tantalum oxide (Ta2O5), titanium, titanium nitride, PEDOT, carbon nanotubes, gold, platinum, iridium, iridium oxide, ruthenium oxide, or an alloy(s) (e.g., an alloy that includes any of the foregoing materials), and/or any suitable combination thereof. In some embodiments, the interconnect band 750 may be configured to extend between analog nodes of a neural device.

FIG. 4B is a schematic diagram of neural device 800 including an interposer 870 coupled to an integrated circuit chip 860 (e.g., an embedded logic die). In some embodiments, the interposer 870 may be a thinned silicon interposer. In some embodiments, the interposer 870 may define a plurality of openings 855 therein. In some embodiments, the interposer 870 may further include one or more electrodes 853 and/or one or more conductive traces 857 disposed on a first surface thereof (e.g., a surface configured to face the brain 802). In some embodiments, the interposer 870 may include one or more nodes. Each node may be coupled to a respective integrated circuit chip 860. The neural device 800 may be configured to be disposed on a surface of a brain of a user such that the at least one electrode 853 contacts the surface of the brain 802 of the user.

In some embodiments, the interposer 870 may include a first encapsulation material 880 disposed around at least a portion of the interposer 870. In some embodiments, the encapsulation material 880 may be disposed on all sides of the interposer (e.g., a bottom side, a top side, and sides of the interposer defining the openings 855). The encapsulation material(s) 880 may be applied to the interposer after the openings 855 are etched out of the interposer 870. In some embodiments, the encapsulation material 880 may cover the conductive trace(s) 857 such that the conductive trace(s) 857 are not exposed to biological material. In some embodiments, the encapsulation material 880 may not cover a first surface of the electrode(s) 853 configured to contact the brain 802 of the patient. In some embodiments, the electrode(s) 853 may include a first conductive material and the trace(s) 857 may include a second conductive material. In some embodiments, the first conductive material and the second conductive material may be the same material. In some embodiments, the first conductive material and the second conductive materials may be different materials. In some embodiments, the second conductive material include for example, tantalum, tantalum oxide (Ta205), titanium, titanium nitride, PEDOT, carbon nanotubes, gold, platinum, iridium, iridium oxide, ruthenium oxide, or an alloy(s) (e.g., an alloy that includes any of the foregoing materials), and/or any suitable combination thereof.

In some embodiments, the interposer 870 (e.g., at a node) may include (i) an electrical contact disposed on a first surface thereof and (ii) an electrical connection 818 extending from the electrical contact to a second surface of the interposer opposite the first surface. In some embodiments, the electrical connection 818 may be a through-silicon-via (TSV). In some embodiments, the electrical connections 818 act as a landing grid (e.g., points of connection) for the integrated circuit chip 860 to couple the integrated circuit chip 860 to the interposer 870. In some embodiments, the electrical connection 818 can extend through the interposer 870 such that the electrical connection 818 is exposed on both sides of the interposer 870. This can allow the integrated circuit chip 860 to be coupled to either side of the interposer (e.g., a side further from the brain 802 as shown or a side closer to the brain 802). In some embodiments, the interposer 870 may form a grid structure with a plurality of nodes at each intersection of the grid. In such embodiments, each node or a subset of the nodes may include electrical connections 818 configured to couple to an integrated circuit chip 860. In some embodiments, the interposer 870 may instead include one or more nodes adjacent to the grid structure, the one or more nodes may be coupled to a respective integrated circuit chip 860.

In some embodiments, the integrated circuit chip 860 may be coupled to the interposer on the first side thereof (e.g., opposite the electrode(s) 853). In some embodiments, the integrated circuit chip 860 may be coupled to the interposer 870 on a second side thereof opposite the first side (e.g., the same side as the electrode(s) 853). In some embodiments, the integrated circuit chip 860 may be disposed on the interposer 870 on the first side and a second integrated circuit chip 860 may be disposed on the second side of the interposer 870.

In some embodiments, the integrated circuit chip 860 may include one or more electrical contacts 862 on a first surface thereof. At least one of the one or more electrical contacts 862 may be configured to electrically connect to the electrical connection 818 of the interposer 870. In some embodiments, the electrical contacts 862 may be coupled to the electrical connection 818 via one or more bump bonds. In some embodiments, the integrated circuit chip 860 may further include one or more encapsulation materials 882 disposed on at least a portion of the integrated circuit chip 860 and electrode contacts 862. In some embodiments, a first encapsulation layer may be disposed on the first surface of the integrated circuit chip 860. In some embodiments, a second encapsulation layer may be disposed over the first encapsulation layer and/or wrapped around a portion of the integrated circuit chip 860. In some embodiments, the integrated circuit chip 860 may optionally include a third encapsulation layer disposed on a second surface opposite the first surface. In some embodiments, the encapsulation layers may include any of the encapsulation materials described herein. In some embodiments, the first encapsulation layer may include at least one of transfer bonded thermal SiO₂ or anodically bonded SiC. In some embodiments, the second encapsulation layer and/or the third encapsulation layer may include at least one of SiC and/or parylene-C. In some embodiments, the interposer 870 may be thinned to a predetermined thickness in a range between about 1 μm and about 40 μm, inclusive of all ranges and subranges therebetween. In some embodiments, the predetermined thickness of the interposer 870 may be between about 5 μm and about 25 μm. In some embodiments, one or more portions of the interposer 870 nay be configured to flex along a curvature of the brain. In some embodiments, a portion of the neural device 800 including the integrated circuit chip 860 coupled to the interposer 870 may be sufficiently rigid to protect the electronics therein.

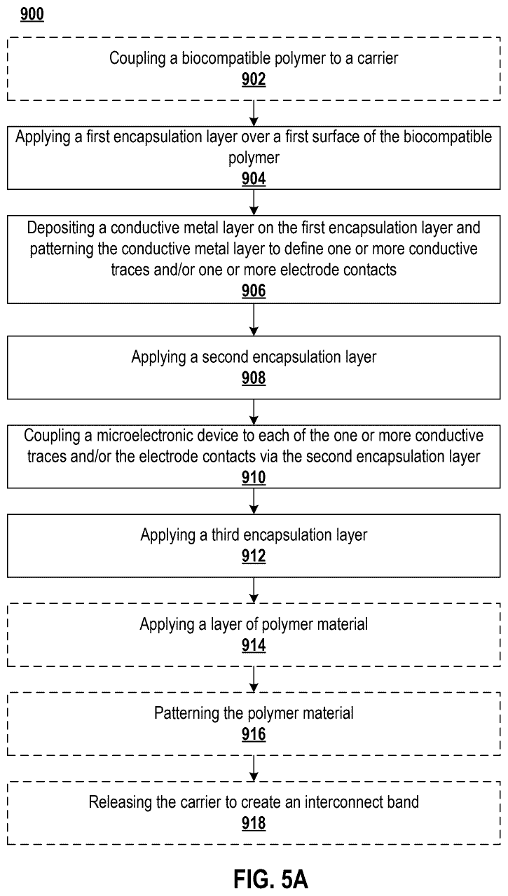

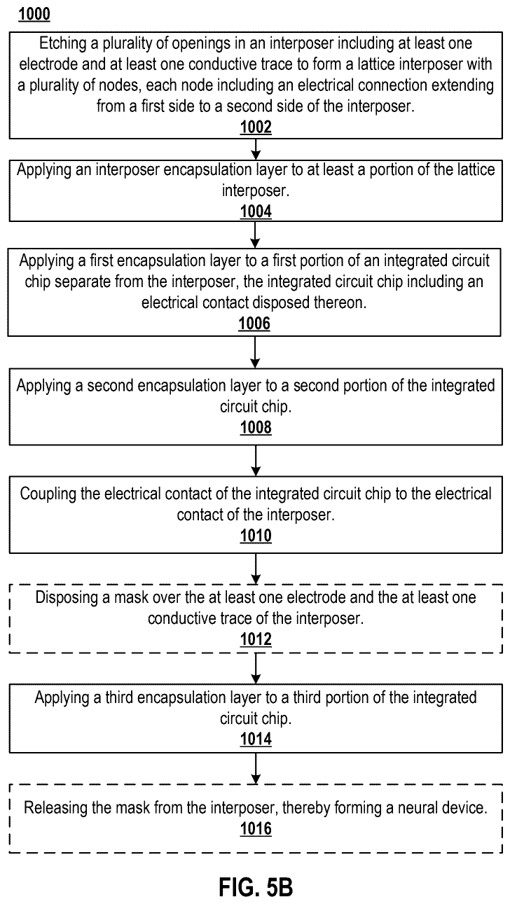

FIG. 5A is a method 900 of manufacturing an interconnect band (e.g., interconnect band 750) of a neural device, according to some embodiments. The method 900 may include, coupling a biocompatible polymer to a carrier, at 902. In some embodiments, the biocompatible polymer can be at least one of LCP, polyimide, and/or silicon (e.g., a silicon interposer). In some embodiments, the carrier may include any suitable material such as, for example, quartz, glass, metal, silicon, and/or ceramic. In some embodiments, the coupling the biocompatible polymer to the carrier may include deposition of the biocompatible polymer via lamination and/or spin coating. In some embodiments, the method 900 may include depositing a metal hard mask on the biocompatible polymer. The depositing the metal hard mask may include performing any suitable method including, for example, physical vapor deposition (PVD), chemical vapor deposition (CVD), atomic layer deposition, pulsed layer deposition, sputtering, or any suitable combination thereof. In some embodiments, the metal hard mask may be deposited via PVD and/or sputtering. In some embodiments, the metal hard mask may include any suitable material such as aluminum (Al). The method 900 may include patterning the hard mask and/or the biocompatible polymer to form one or more recesses. The patterning may include any suitable method such as, for example, electron-beam evaporation, electroplating, electroless plating, lithography, photolithography, plasma etching, wet etching, dry etching, reactive-ion etching, sputtering, and/or lift-off processes. In some embodiments, the patterning the metal hard mask may include performing wet etching. In some embodiments, the patterning the biocompatible polymer may include performing dry etching.

At 904, the method 900 may include applying a first encapsulation layer over a first side of the biocompatible polymer. In some embodiments, the first encapsulation layer may be deposited on the metal hard mask and configured to cover surfaces defining the recesses. The first encapsulation layer may be deposited such that all exposed surfaces of the metal hard mask and/or the biocompatible polymer are covered while maintaining the recesses formed in biocompatible polymer. In some embodiments, the first encapsulation layer may any suitable material such as, for example, silicon carbide, polyimide (PI), parylene-C, silicon dioxide, liquid-crystal polymer (LCP), silicone, photoresist (e.g., SU-8), alumina, ceramic-polymer composites, compounds such as polymers with silicon carbide (SiC) partially diffused therein, doped SiC, or any other suitable biocompatible material. In some embodiments, method 900 may further include patterning the first encapsulation layer. In some embodiments, the patterning may remove a portion of the first encapsulation layer defining a bottom of the recesses such that a portion of the carrier is exposed. The patterning may include any suitable patterning method such as dry etching.

At 906, the method 900 may include depositing a conductive metal layer on the first encapsulation layer and patterning the conductive metal layer to define one or more conductive traces and/or one or more electrode contacts, at 906. In some embodiments, the depositing the conductive metal layer on the first encapsulation layer may include metallization via PVD, CVD, sputtering, e-beam evaporation, electroplating, electroless plating, etc. The first conductive metal layer may include any suitable material such as, for example, tantalum, tantalum oxide (Ta2O5), titanium, titanium nitride, PEDOT, carbon nanotubes, gold, platinum, iridium, iridium oxide, ruthenium oxide, or an alloy(s) (e.g., an alloy that includes any of the foregoing materials). In some embodiments, the one or more electrodes and the one or more electrical traces may include the same material. In some embodiments, the one or more electrodes may include a first material, and the one or more electrical traces may include a second material different than the first material. In some embodiments, patterning can include, for example, sputtering, e-beam evaporation, electroplating, electroless plating, lithography, photolithography, plasma etching, wet etching, reactive-ion etching, lift-off processes, or a suitable combination thereof. In some embodiments, the patterning of the conductive metal layer may include wet etching.

At 908, the method 900 may include applying a second encapsulation layer over the first side of the biocompatible polymer such that the patterned conductive metal layer is at least partially covered. The second encapsulation layer may be disposed using any suitable method, such as any of the deposition methods described herein. In some embodiments, the method 900 may further include patterning the second encapsulation layer such that the second encapsulation layer defines one or more openings over the one or more electrodes. Therefore, the one or more electrodes may be exposed such that they can be electrically connected to a microelectronic device. In some embodiments, the patterning the second encapsulation layer may include dry etching the second encapsulation layer.

At 910, the method 900 may include coupling a microelectronic device to at least one of the one or more conductive traces and/or the one or more electrodes through the second encapsulation layer (e.g., through the openings etched in the second encapsulation layer). In some embodiments, the microelectronic devices may be coupled to the electrode contacts via bumping (e.g., bump bonds) and/or through a flip-chip process. At 912, the method may further include applying a third encapsulation layer over the microelectronic device. The applying the third encapsulation layer may include covering surfaces of the microelectronic devices. The third encapsulation layer may be applied via any suitable method such as any of the deposition methods described herein. In some embodiments, the third encapsulation layer may be applied via CVD. In some embodiments, the first encapsulation layer, the second encapsulation layer, and the third encapsulation layer may include the same material. In some embodiments, any one of the first encapsulation layer, the second encapsulation layer, and/or the third encapsulation layer may include a different material. In some embodiments, the first encapsulation layer, the second encapsulation layer, and/or the third encapsulation layer may include any suitable material such as, for example, silicon carbide, polyimide (PI), parylene-C, silicon dioxide, liquid-crystal polymer (LCP), silicone, photoresist (e.g., SU-8), alumina, ceramic-polymer composites, compounds such as polymers with silicon carbide (SiC) partially diffused therein, doped SiC, or any other suitable biocompatible material. In some embodiments, at least one of the first encapsulation layer, the second encapsulation layer, or the third encapsulation layer includes silicon carbide (SiC).

In some embodiments, the method may include patterning the third encapsulation layer (e.g., to the metal hard mask). In some embodiments, the metal hard mask may act as an etch stop to prevent etching of the biocompatible polymer. In some embodiments, the etching the third encapsulation layer may include wet and/or dry etching steps. In some embodiments, the method 900 may include removing the metal hard mask. In some embodiments, the metal hard mask may be wet etched. At 914, the method includes applying a layer of polymer material. In some embodiments, the polymer material may be the same as the biocompatible polymer. In some embodiments, the biocompatible polymer may be disposed through lamination and/or spin coating. At 916, the method may include patterning the biocompatible polymer such that an opening is defined between the one or more conductive traces and/or the electrode contacts. In some embodiments, the method 900 may include releasing the carrier to create the interconnect band, at 918.

FIG. 5B is a method 1000 of manufacturing a neural device including an interposer coupled to an integrated circuit, according to some embodiments. In some embodiments, the method 1000 may include etching a plurality of openings in an interposer (e.g., a thinned silicon interposer), at 1002. The thinned silicon interposer may include at least one electrode and at least one conductive trace and may form a lattice or mesh structure with a plurality of nodes, each node including an electrical connection extending from a first side of the interposer to a second sider of the interposer. In some embodiments, the method 1000 may include applying an interposer encapsulation layer to at least a portion of the lattice interposer, at 1004. In some embodiments, the interposer encapsulation material may include any suitable encapsulation material, such as for example, SiC and/or parylene-C. In some embodiments, the method 1000 may include applying a first encapsulation layer to a first portion of an integrated circuit chip separate from the interposer, at 1006. The integrated circuit chip may include an electrical contact disposed thereon. The integrated circuit chip may further include a bump bond/reflow extending from the electrical contact. In some embodiments, the method may include applying a second encapsulation layer to a second portion of the integrated circuit chip, at 1008. In some embodiments, the first encapsulation layer may include a first encapsulation material and the second encapsulation layer may include a second encapsulation material different than the first encapsulation material. In some embodiments, the first encapsulation material may include SiO2 and/or SiC. In some embodiments, the second encapsulation material may include SiC and/or parylene-C. In some embodiments, the bump bond may extend through the first encapsulation layer and the second encapsulation layer of the integrated circuit chip.

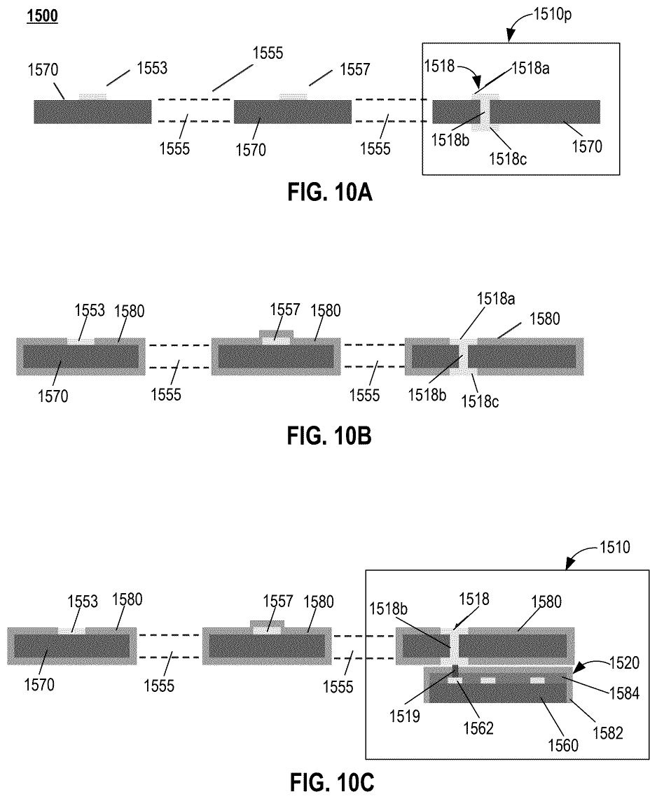

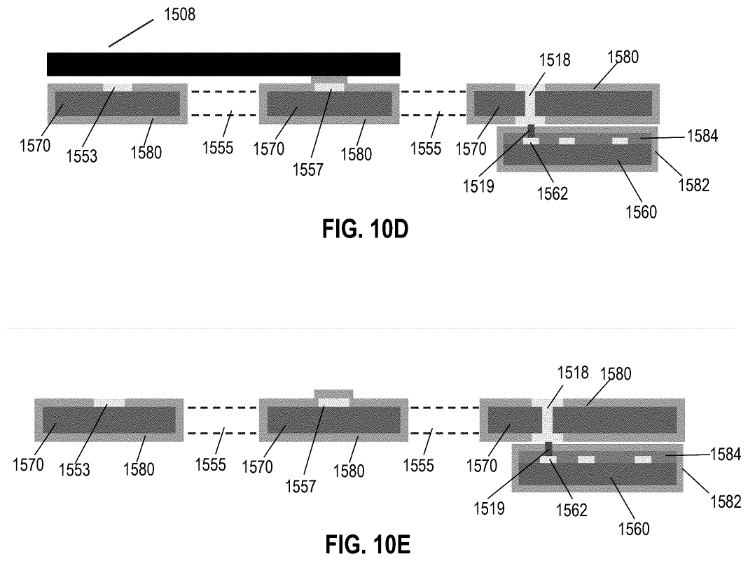

At 1010, the method 1000 includes coupling the electrical contact of the integrated circuit chip to the electrical contact of the interposer (e.g., via the bump bond). In some embodiments, the first encapsulation layer and/or the second encapsulation layer can be disposed on the integrated circuit chip before the integrated circuit chip is coupled to the interposer. In some embodiments, the method 1000 may further include disposing a mask over the at least one electrode and the at least one conductive trace of the interposer. At 1014, the method includes applying a third encapsulation layer to a third portion of the integrated circuit chip. In some embodiments, the third encapsulation layer can be disposed on the integrated circuit chip after the integrated circuit chip is coupled to the interposer. In some embodiments, the mask may protect the electrode during deposition of the third encapsulation layer. In some embodiments, the first, second, and third encapsulation layers may cover all sides of the integrated circuit chip. In some embodiments, the third encapsulation layer may include the same encapsulation material as the second encapsulation layer. The method 1000 may further include releasing the mask from the interposer, thereby forming the neural device, at 1016. Further details of the method 1000 are described in FIGS. 10A-10E, and therefore are not described herein with respect to FIG. 5B.

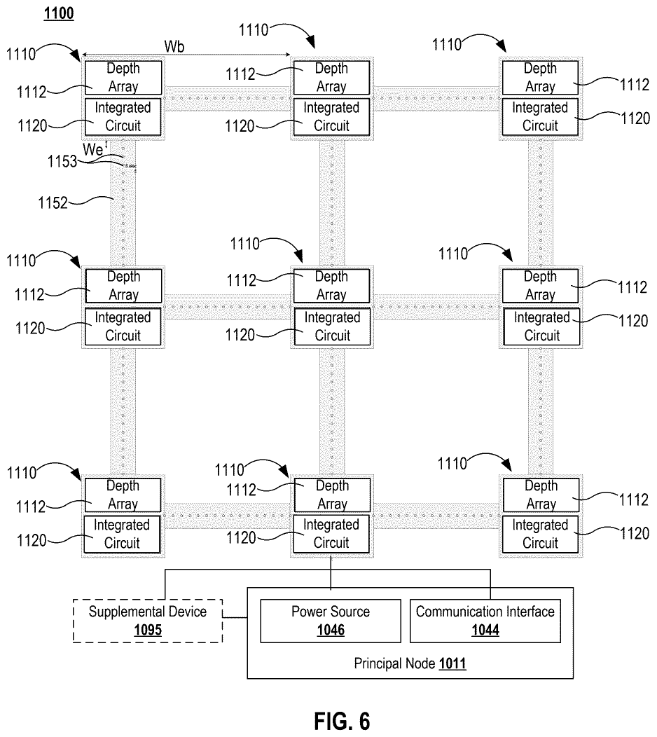

FIG. 6 is a neural device 1100, according to some embodiments. As shown, the neural device 1100 includes a plurality of nodes 1110 (e.g., islands, intersections, hubs, connection points, etc.) each including a depth electrode array 1112 and associated integrated circuit 1120 (Application Specific Integrated Circuit (ASIC)). Each node 1110 may further include transistors. As shown, the plurality of nodes 1110 are connected via connectors 1152 (e.g., interconnection bands). The interconnect bands 1152 may include one or more surface electrodes 1153 (e.g., μECOG) disposed thereon. The interconnect bands 1152 may include bi-directional data lines extending adjacent to the surface electrodes 1153. In some embodiments, the data lines may be coupled to a central or principal node 1011 including a communication interface 1044 and/or a power source 1046 (e.g., a remote power source). In some embodiments, the principal node 1011 may be disposed at a terminal end of the neural device 1000, as shown. In some embodiments, the neural device 1100 may further be configured to communicate with a supplemental device 1095 such as an off-brain device (e.g., other implants, sensors, etc.). In some embodiments, the principal node 1011 may be configured to provide wireless communication and power to the plurality of nodes 1110. In some embodiments, the principal node 1011 and/or the interconnect bands 1052 may experience noise due to high data rates that scales with the number of nodes 1110 and/or size of the neural device 1100. Therefore, analog and digital data lines may be separated, as described in FIGS. 1, 2, 3A-3C, and 7A-8C.

The neural device 1100 may allow whole-brain LFP and AP recording and stimulation. In some embodiments, the neural device 1100 may be formed from a single wafer (e.g., a biocompatible wafer). The neural device 1100 may be fabricated on a single wafer, backgrinded (e.g., ground or thinned from a backside of the wafer) to minimal thickness and etched in between the interconnection bands 1152 to provide flexibility and proper attachment on brain shape. In some embodiments, the neural device 1100 may be formed from a biocompatible material, and the openings defined by the interconnect bands 1152 may be removed such that the neural device forms a mesh or lattice structure. In some embodiments, the neural device may have a square configuration in which the interconnect bands 1152 define square openings. The plurality of nodes 1110 may be disposed at each vertex of the square, as shown. Each square defined by the interconnect bands may be referred to as a tile. In some embodiments, the neural device may include a plurality of tiles coupled together. As shown, the neural device 1100 includes 4 tiles coupled together to form a 2×2 array of tiles. In some embodiments, the 2×2 array of tiles may have a width between about 1 cm to about 3 cm and a length between about 1 cm to about 3 cm, inclusive of all ranges and subranges therebetween.

The depth array 1112 may include a 9 by 4 electrode array including a total of 36 electrodes. In some embodiments, the neural device may include a total number of depth electrodes between about 36 depth electrodes and about 81,920 depth electrodes, inclusive of all ranges and subranges therebetween. In some embodiments, the neural device may include up to about 165,000 electrodes, for example when the neural device does not include any recesses (e.g., openings, perforations, through-holes, vias, etc.). In some such embodiments, the neural device may include between about 82,000 electrodes and about 165,000 electrodes, or between about 150,000 electrodes and about 200,000 electrodes. In some embodiments, the neural device may include up to about 82,000 electrodes, for example when the neural device has about a 50% fill ratio (e.g., where 50% of the material is removed to define openings). In some such embodiments, the neural device may include between about 10,000 electrodes and about 100,000 electrodes, or between about 50,000 electrodes and about 100,000 electrodes, or between about 25,000 electrodes and about 75,000 electrodes. In some embodiments, the depth array 1112 may include a UTAH array. In some embodiments, the depth array may have a width of about 3.6 mm and length of about 1.6 mm. In some embodiments, the integrated circuit 1120 may include an ASIC with a length of about 3.6 mm and a length of about 1.8 mm. In some embodiments, the ASIC may have a total area of between 1 mm² to 10 mm². In some embodiments, the ASIC may have a total area of about 6.5 mm². In some embodiments, the surface electrodes 1153 may have a distance. We therebetween in a range of about 100 μm to about 1000 μm, inclusive of all ranges and subranges therebetween. In some embodiments, the distance We between the surface electrodes 1153 may be in a range of about 200 μm to about 500 μm, inclusive of all ranges and subranges therebetween. (between electrodes). In some embodiments, each interconnect band 1152 may include between about 1 surface electrode and about 100 surface electrodes, inclusive of all ranges and subranges therebetween. In some embodiments, the interconnect band 1152 may include between about 10 surface electrodes and about 20 surface electrodes, inclusive of all ranges and subranges therebetween. As shown, the neural device 1110 may include about 324 depth electrodes in total and 192 surface electrodes for a total of 516 electrodes in total.

In some embodiments, each interconnect band 1052 may include one or more electrode lines (e.g., traces), one or more signal lines (e.g., traces), and/or one or more power lines (e.g., traces). In some embodiments, a minimum width of each line may be greater than about 10 um, about 15 um, about 20 um, about 25 um. In some embodiments, a minimum spacing between adjacent lines may be greater than about 1 um, greater than about 5 um, greater than about 10 um, greater than about 15 um, greater than about 20 um. In some embodiments, the minimum pitch of the lines may be greater than about 10 um, about 15 um, about 20 um, about 25 um. In some embodiments, a width of the interconnect band 1052 may be no greater than about 100 um, no greater than about 125 um, no greater than about 150 um, no greater than about 175 um, no greater than about 200 um. In some embodiments, the integrated circuit 1120 may be configured to accommodate 1 depth array 1112 and a number of surface electrodes on two interconnect bands. Certain aspects of the neural device 600 may be structurally and/or functionally similar to any of the neural devices described herein, and therefore certain aspects of the neural device 1100 are not described herein with respect to FIG. 6.

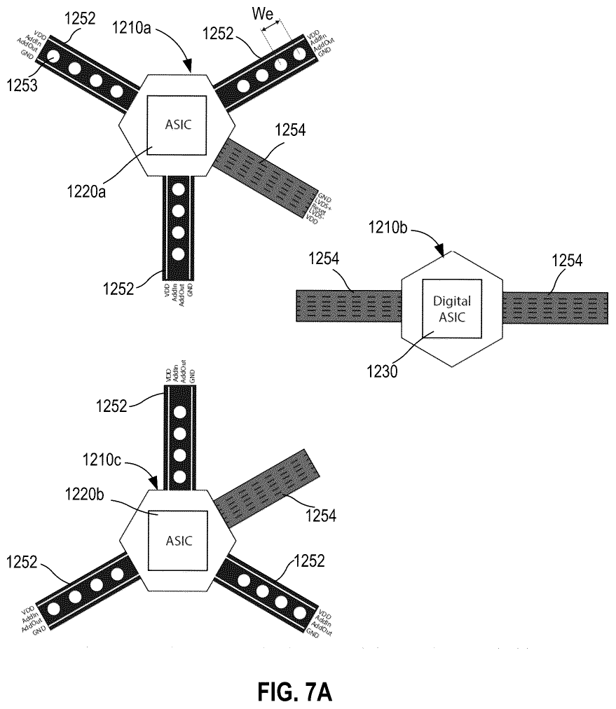

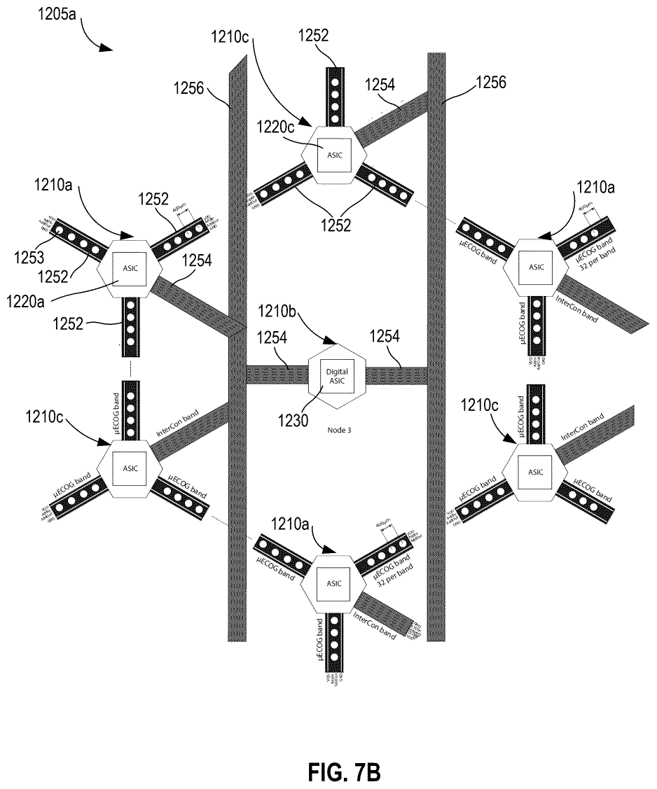

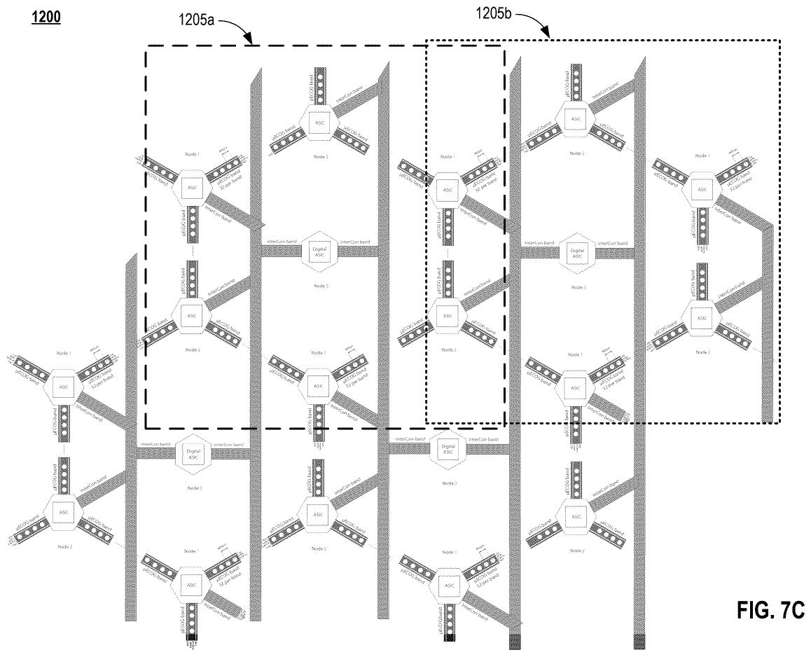

FIG. 7A shows a first node 1210a from the first set of nodes, a second node 1210b from the second set of nodes, and a third node 1210c from the third set of nodes, according to some embodiments. As shown, the first node 1210a and the second node 1210c may be configured for analog stimulation and recording. The first node 1210a and the second node 1210c may include an integrated circuit (e.g., ASIC) 1220a, 1220c including analog circuitry (e.g., analog front end for controlling one or more electrodes). The integrated circuit 1220a 1220c may be structurally and/or functionally to the analog microelectronics described in FIGS. 2 and 3A.

The first node 1210a and the second node 1210c may each include three analog bands 1252, 1254 each extending from a side of thereof. The analog bands 1252 may each include a plurality of surface electrodes (e.g., μECOG) 1253 disposed along a surface of the analog band 1252. The plurality of surface electrodes 1253 may be distributed along a length of each analog band 1252. In some embodiments, the analog band 1252 may include any suitable number of surface electrodes 1253. In some embodiments, the analog band 1252 may include between about 2 electrodes and about 64 electrodes, inclusive of all ranges and subranges therebetween. In some embodiments, the analog band 1252 may include about 32 electrodes, inclusive of all ranges and subranges therebetween. In embodiments, a distance between adjacent surface electrodes 1253 may be in a range between about 200 μm to 800 μm, inclusive of all range and subranges therebetween. In some embodiments, the analog bands 1252 may include circuitry thereon or extending therethrough for recording analog neural signals and stimulating neurons.

The first node 1210a and the second node 1210c may each include a hexagon shape with a first side, a second side, a third side, a fourth side, a fifth side, and a sixth side. In some embodiments, the first node 1210a may include a first band extending from the first side (e.g., a top left side) of the hexagonal shape, a second band extending from the third side (e.g., top right side) of the hexagonal shape, and a third analog band extending from the fifth side (e.g., a bottom side) of the hexagonal shape. The second node 1210c may include a first analog band extending from the second side (e.g., the top side), a second analog band extending from the fourth side (e.g., the bottom right side), and a third analog band extending from the sixth side (e.g., the bottom left side). The first node 1210a and the second node 1210b may each include a digital band 1254. In some embodiments, the digital band 1254 may extend from a side of the first node 1210a between the second analog band and the third analog band. In some embodiments, the digital band 1254 of the second node 1210c may extend from a side of the second node 1210c between the first analog band and the second analog band. In some embodiments, the first node 1210a and the second node 1210c may be mirror images of one another. In some embodiments, analog bands 1252 can extend from the analog nodes 1210a, 1210b defining equal angle or spacing therebetween.

The third node 1210b may include two digital bands 1254 extending therefrom. For example, a first digital band 1254 may extend from a first side of the second node 1210b and a second digital band 1254 may extend from a second side opposite the first side. In some embodiments, the third node 1210b may include a digital circuit (e.g., a digital ASIC) 1230 configured to perform one or more signal processing functions.

The third node 1210b may not feature analog bands and may be configured for signal processing for real-time closed loop application, data compression, machine learning (ML), and/or RF. The third node 1210b may be configured to perform auto-addressing of the entire neural device. For example, the third node 1210b may run software and provide clockwise unique ID to each title, node, and/or stim/rec channel. The auto-addressing algorithm may identify neuron spikes and the corresponding electrode at which the neuron spikes are detected.

FIGS. 7B-7C show a neural device including a plurality of nodes 1210a, 1210b, 1210cc connected by a plurality of interconnect bands 1252, 1254, 1256, according to some embodiments. In some embodiments, the neural device may include a biocompatible polymer substrate, the biocompatible polymer substrate forming the plurality of nodes and interconnect bands and defining a plurality of openings therein. In some embodiments, the neural device may be encapsulated with at least one encapsulation material, as described in above in FIGS. 4A-4B.

A subset of nodes may be coupled together to form a tile 1205a, 1205b. FIG. 7B shows a tile 1205a. In some embodiments, the tile 1205a may include three of the first type of nodes (type 1 nodes) 1210a, three of the second type of nodes (type 2 nodes) 1210c, and one node of the third type of nodes (type 3 nodes) 1210b. The type 1 nodes 1210a may be connected to the type 2 nodes 1210c by analog bands 1252. The type 1 nodes 1210a and the type 2 nodes 1210c may be coupled to the type 3 nodes 1210b via digital bands 1254 and digital bands 1256. The six analog nodes 1210a, 1210c may be arranged in a hexagonal shape with the analog bands 1252 forming each side of the hexagon. Each tile 1205a, 1205b may include vertical high-speed digital bands 1256 (e.g., digital highways) extending from a first end of the tile 1205a, 1205b to a second end of the tile 1205a, 1205b. The digital bands 1254 (e.g., low or medium speed digital bands) of the first node 1210a and the second node 1210c may be electrically connected to the one of the high-speed digital bands 1256 (e.g., digital highways). The third node 1210b may be disposed between the digital highways 1256, and the first digital band 1254 of the third node 1210b may couple to the first digital highway 1256 and the second digital band 1254 of the third node 1210b may couple to the second digital highway 1256. The organization of the bands in each tile physically separates the digital lines 1254, 1256 and the analog lines 1252 for improved signal quality. Digital lines can inject noise onto adjacent lines which can degrade the quality of recorded data (e.g., recorded analog neural signals). Physical spacing between the low noise analog lines and the high-speed digital lines can reduce this noise and improve signal quality.

As shown in FIG. 7C the neural device 1200 may include a plurality of tiles 1205a, 1205b coupled together. As shown, each side of a given tile 1205a overlaps with a side of an adjacent tile 1205b. The plurality of tiles 1205a, 1205b may form a honeycomb pattern or a lace pattern. Certain aspects of the neural device 700 may be structurally and/or functionally similar to any of the neural devices described herein, and therefore certain aspects of the neural device 1200 are not described herein with respect to FIGS. 7A-7C.

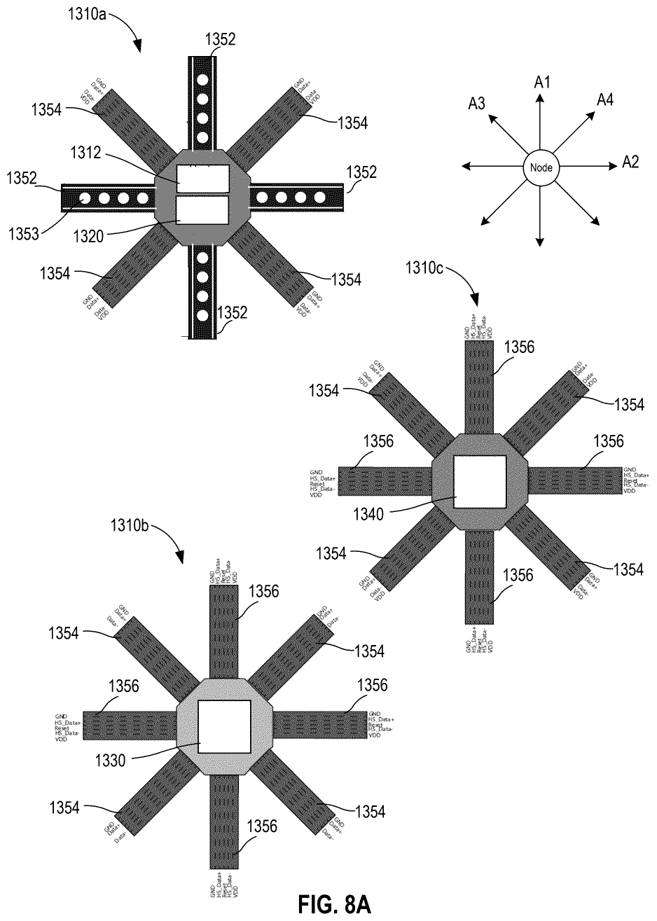

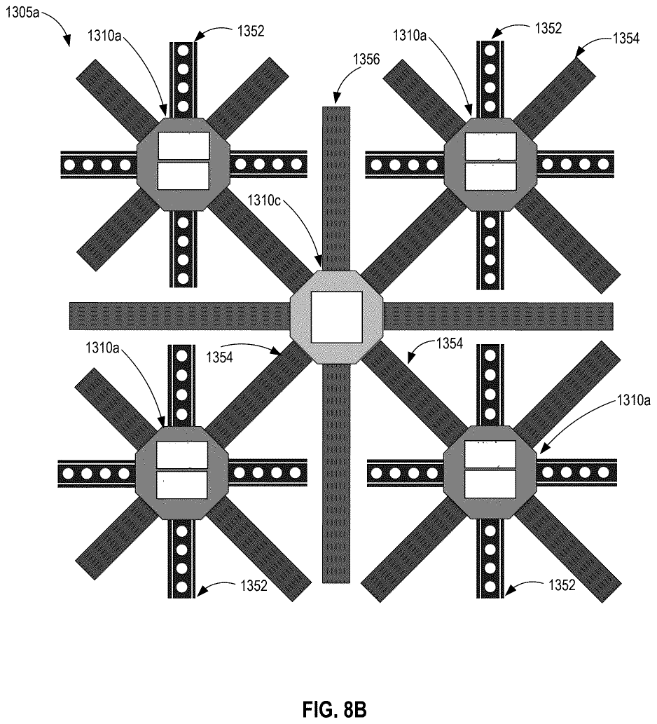

FIG. 8A shows a first node, 1310a, a second node 1310b, and a third node 1310c of the neural device of FIGS. 8A-8B, according to some embodiments. The first node 1810a may be configured as an analog node, the second node 1310b may be configured as a digital node, and the third node 1810c may be configured as a communication node. As shown, each node 1310a, 1310b, 1310c may include an octagonal shape including eight sides. Each analog node 1310a can include bands from a first subset of bands (e.g., analog bands 1352) extending therefrom and bands from a second subset of bands (e.g., medium-speed digital bands 1354). The first subset of bands can be configured to connect the analog node 1310a to neighboring analog nodes. The second subset of bands can be configured to connect the analog node 1310a to a nearby digital node 1310c. Each analog node 1310a may include 4 analog bands 1352 extending therefrom. The 4 analog bands 1352 may extend along a first axis A1 and along a second axis A2 perpendicular to the first axis. In some embodiments, each analog band 1352 may include about 4 surface electrodes 1353 for a total of 16 surface electrodes 1353 per analog circuit. Although shown as including 4 surface electrodes 1353, it should be appreciated that the analog band 1352 may include any suitable number of electrodes. In some embodiments, the surface electrodes 1353 may include μECOG electrodes. The analog node 1310a may further include 4 medium-speed digital bands 1354 (e.g., the second subset of bands) extending therefrom. The 4 medium-speed digital bands 1354 may extend along a third axis A3 and a fourth axis A4 perpendicular to the third axis A3. The third axis A3 and the fourth axis A4 may be different than the first axis A1 and the second axis A2.

The digital node 1310b can include bands from the second subset of bands (e.g., the medium-speed digital bands) 1354 from a third subset of bands (e.g., high-speed digital bands) 1356. Medium-speed digital bands as used herein may refer to digital bands having a transmission rate below about 5 megahertz (MHz). High-speed digital bands as used herein may refer to digital bands having a transmission rate greater than about 5 MHz. In some embodiments, the second subset of bands 1354 can be configured to connect the digital node 1310b to nearby analog bands 1310a, and the third subset of bands 1356 can be configured to connect the digital node 1310b to neighboring communication nodes 1310c. The digital node 1310b may include 4 medium-speed digital bands 1354 and 4 high-speed data lines1356 (e.g., the third subset of bands). The 4 high-speed data lines 1365 may extend along the first axis A1 and the second axis A2, and the 4 medium-speed digital bands 1354 may extend along the third axis A3 and the fourth axis A4. The communication node 1310c may include four medium speed bands and four high-speed data lines. The 4 medium-speed digital bands 1354 may extend along the third axis A3 and the fourth axis A4, and the 4 high-speed digital bands 1356 may extend along the first axis A1 and the second axis A2. The communication node 1310c may provide analog power through the medium-speed bands 1354 and digital power through the high-speed data lines 1356.

FIG. 8B shows a tile 1305a of the neural device including a plurality of nodes 1310a. 1310c coupled together by a plurality of interconnect bands 1352, 1354, 1356. As shown, the tile 1305a includes a digital node 1310c disposed in a center of and connected to 4 analog nodes 1310a. The analog interconnect bands 1352 may define a square or rectangular shape with each analog node 1310a disposed at a vertex of the square or rectangle shape. The digital node 1310c may be disposed at or near a center point of the square or rectangle shape. When connecting multiple tiles 1305a, 1305b together for a large arrangement, the communication nodes 1310c can be added in between tiles 1305a, 1305b, as shown in FIG. 8C. Certain aspects of the neural device 1300 may be structurally and/or functionally similar to any of the neural devices described herein, and therefore certain aspects of the neural device 800 are not described herein with respect to FIGS. 8A-8C.

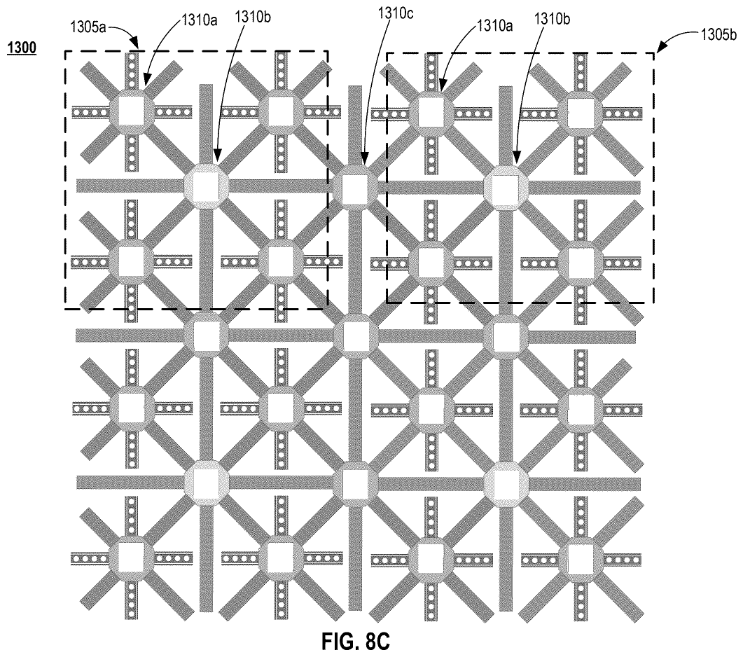

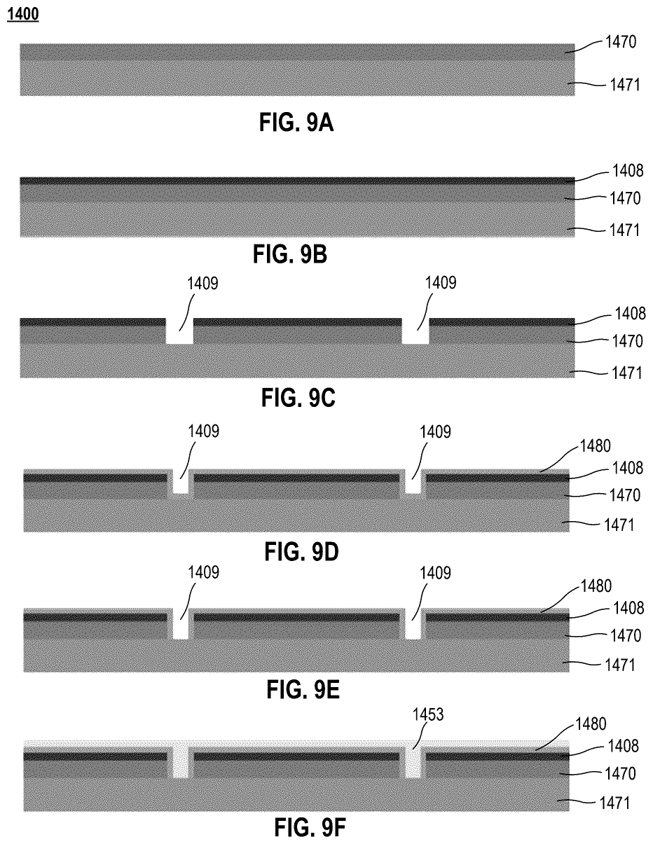

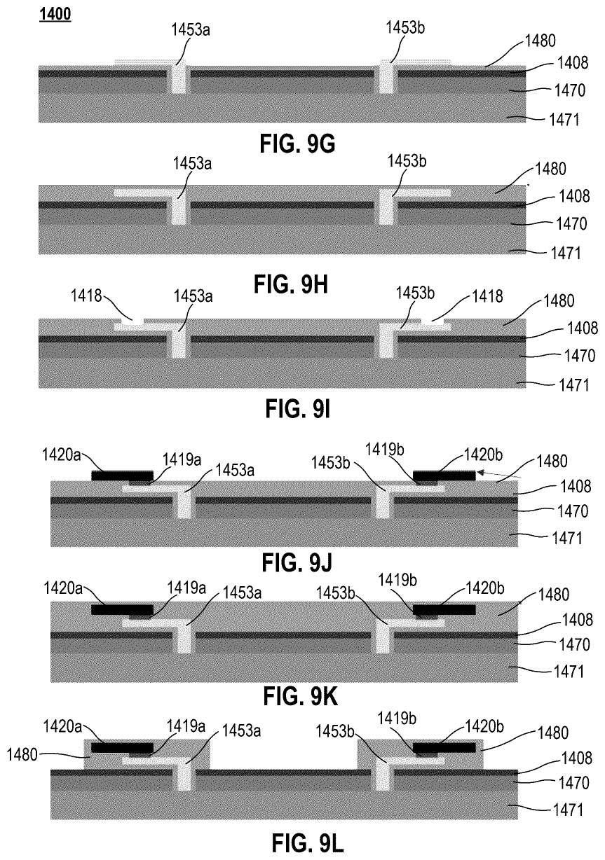

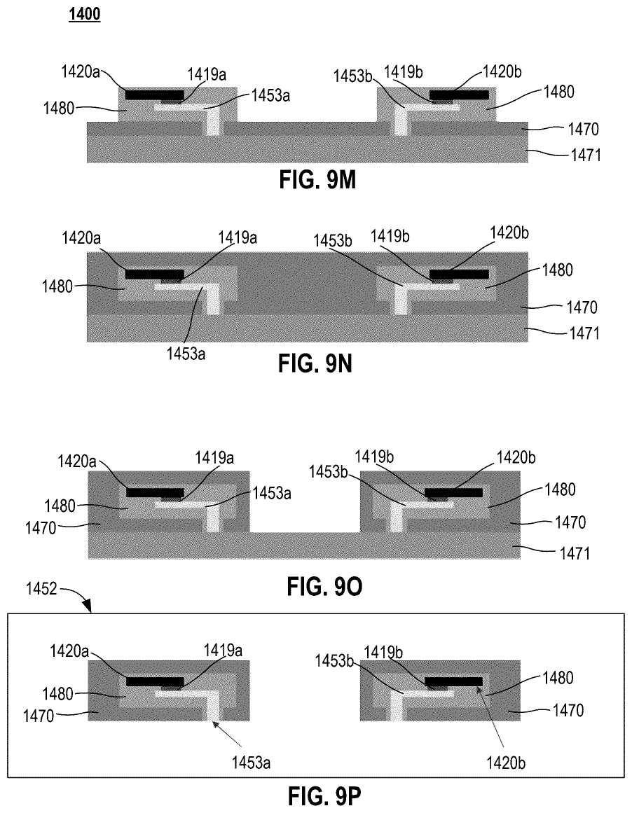

FIGS. 9A-9P show a method 1400 of manufacturing an interconnect band 1352 (e.g., an analog interconnect band) of a neural device, according to some embodiments. As shown in FIG. 9A, the method 1400 may include coupling a biocompatible polymer 1470 to a carrier substrate 1471. The coupling the biocompatible polymer 1470 to the carrier substrate 1471 may include deposition of the biocompatible polymer 1470 via lamination and/or spin coating. In some embodiments, the biocompatible polymer 1470 may include at least one of polyimide or LCP. As shown in FIG. 9B, the method 1400 may deposit a metal hard mask 1408 onto the biocompatible polymer 1470. The depositing the metal hard mask 1408 may include performing any suitable method including, for example, physical vapor deposition (PVD), chemical vapor deposition (CVD), atomic layer deposition, pulsed layer deposition, sputtering, or any suitable combination thereof. In some embodiments, the metal hard mask 1408 may be deposited via PVD and/or sputtering. In some embodiments, the metal hard mask 1408 may include any suitable material such as aluminum (A1), chrome (Cr), gold (Au), and/or titanium (Ti).

As shown in FIG. 9C, method 1400 may include patterning the metal hard mask 1408 and/or biocompatible polymer 1470 to form one or more recesses 1409. The patterning may include any suitable method such as, for example, beam evaporation, electroplating, electroless plating, lithography, photolithography, plasma etching, wet etching, dry etching, reactive-ion etching, and/or lift-off processes. In some embodiments, the patterning the metal hard mask 1408 may include performing wet etching. In some embodiments, the patterning the biocompatible polymer 1470 may include performing dry etching. As shown in FIG. 9D, method 1400 may include disposing a first encapsulation layer 1480 over at least a portion of the patterned metal hard mask 1408 and biocompatible polymer 1470 to form a first intermediate structure. In some embodiments, the depositing the first encapsulation layer 1480 may include any suitable method such as, for example, PVD, CVD, atomic layer deposition, pulsed laser deposition, sputtering, or any suitable combination thereof. In some embodiments, the deposing the first encapsulation layer 1480 may include performing chemical vapor deposition. The first encapsulation layer 1480 may be deposited such that all exposed surfaces of the metal hard mask 1408 and/or the biocompatible polymer 1470 are covered while maintaining the recesses 1409 in the biocompatible polymer 1470. In some embodiments, the first encapsulation layer 1480 may have a thickness in a range of about 10 nm to about 50 μm, inclusive of all ranges and subranges therebetween. The first encapsulation layer 1480 may include any suitable material such as, for example, silicon carbide, polyimide (PI), parylene-C, silicon dioxide, liquid-crystal polymer (LCP), silicone, photoresist (e.g., SU-8), alumina, ceramic-polymer composites, compounds such as polymers with silicon carbide (SiC) partially diffused therein, doped SiC, or any other suitable biocompatible material. As shown in

As shown in FIG. 9E, the method 1400 may include patterning the first encapsulation layer 1480. The patterning the first encapsulation layer 1480 may include removing a portion of the first encapsulation layer 1480 defining a bottom of the cavities 1409 such that the carrier substrate 1471 is exposed. In some embodiments, the patterning may include any suitable patterning method such as dry etching. As shown in FIG. 9F, the method 1400 may include metallization (e.g., PVD, CVD, sputtering, e-beam evaporation, electroplating, electroless plating, etc.) of the first encapsulation layer 1480 such that a conductive metal layer 1453 is deposited on the first encapsulation layer 1480. The first conductive metal layer 1453 may include any suitable material such as, for example, tantalum, tantalum oxide (Ta2O5), titanium, titanium nitride, PEDOT, carbon nanotubes, gold, platinum, iridium, iridium oxide, ruthenium oxide, or an alloy(s) (e.g., an alloy that includes any of the foregoing materials). The method 1400 may include patterning the conductive metal layer 1453 to define one or more electrode contacts and/or one or more electrical traces. As shown in FIG. 9G, the conductive metal layer may be patterned to form a first electrode contact 1453a and a second electrode contact 1453b and/or one or more electrical traces (not shown in FIG. 9G.). In some embodiments, the one or more electrodes and the one or more electrical traces may include the same material. In some embodiments, the one or more electrodes may include a first material, and the one or more electrical traces may include a second material different than the first material. In some embodiments, patterning can include, for example, sputtering, e-beam evaporation, electroplating, electroless plating, lithography, photolithography, plasma etching, wet etching, reactive-ion etching, lift-off processes, or a suitable combination thereof. In some embodiments, the patterning of the conductive metal layer 1453 may include wet etching.

As shown in FIG. 9H, the method 1400 may include applying a second encapsulation layer 1480 to at least a portion of the first intermediate structure to form a second intermediate structure. The second encapsulation layer 1480 may be disposed over the first electrode 1453a and the second electrode 1453b to encapsulate the first electrode 1453a and the second electrode 1453b. The second encapsulation layer 1480 may be disposed using any suitable method, such as any of the deposition methods described herein. As shown in FIG. 91, the method 1400 may include patterning the second encapsulation layer 1480. The second encapsulation layer 1480 may be patterned such that the second encapsulation layer defines one or more openings over the one or more electrodes (e.g., first electrode 1453a and second electrode 1453b) such that a portion of the one or more electrodes is exposed. In some embodiments, the patterning the second encapsulation layer 1480 may include dry etching the second encapsulation layer.

The method 1400 may include coupling a microelectronic device from a set of at least one microelectronic devices 1420a, 1420b to each of the electrode contacts 1453a, 1453b and/or to the one or more electrical traces to form a third intermediate structure. As shown in FIG. 9J, a first microelectronic device 1420a is coupled to the first electrode contact 1453a, and the second microelectronic device 1420b is coupled to the second electrode contact 1453. The microelectronic devices 1420a, 1420b may be coupled to the electrode contacts 1453a, 1453b through the encapsulation layer(s) 1480. In some embodiments, the microelectronic devices 1420a, 1420b may be coupled to the electrode contacts 1453a, 1453b via bumping (e.g., bump bonds 1419a, 1419b) and/or through a flip chip process.

As shown in FIG. 9K, the method 1400 may include applying a third encapsulation layer 1480 on at least a portion of the third intermediate structure, thereby producing a fourth intermediate structure. The applying the third encapsulation layer 1480 may include covering surfaces of the microelectronic devices 1420a, 1420b. The third encapsulation layer 1480 may be applied via any suitable method such as any of the deposition methods described herein. In some embodiments, the third encapsulation layer 1480 may be applied to the third intermediate structure via CVD. In some embodiments, the first encapsulation layer, the second encapsulation layer, and the third encapsulation layer may include the same material (as shown in FIGS. 9A-9P). In some embodiments, any one of the first encapsulation layer, the second encapsulation layer, and/or the third encapsulation layer may include a different material. In some embodiments, the first encapsulation layer, the second encapsulation layer, and/or the third encapsulation layer may include any suitable material such as, for example, silicon carbide, polyimide (PI), parylene-C, silicon dioxide, liquid-crystal polymer (LCP), silicone, photoresist (e.g., SU-8), alumina, ceramic-polymer composites, compounds such as polymers with silicon carbide (SiC) partially diffused therein, doped SiC, or any other suitable biocompatible material. In some embodiments, at least one of the first encapsulation layer, the second encapsulation layer, or the third encapsulation layer includes silicon carbide (SiC).

As shown in FIG. 9L, the method 1400 may include patterning the fourth intermediate structure until reaching the hard metal mask 1408. The hard metal mask 1408 may act as an etch stop to prevent further etching of the fourth intermediate structure. In some embodiments, the etching the fourth intermediate structure may include wet and/or dry etching steps. In some embodiments, the etching the fourth intermediate structure may physically separate the first electrode contact 1453a with the first microelectronic device 1420a from the second electrode contact 1453b with the second microelectronic device 1420b. As shown in FIG. 9M, the method 1400 may include removing the metal hard mask 1408. In some embodiments, the metal hard mask 1408 may be removed through wet etching, for example.

As shown in FIG. 9N, the method 1400 may include applying a layer of biocompatible polymer 1470 (e.g., CLP and/or polyimide) on at least portion of the structure with the metal hard mask removed, thereby producing a fifth intermediate structure. In some embodiments, the deposition of the biocompatible polymer 1470 may include any suitable method (e.g., any method described herein). In some embodiments, the deposition of the biocompatible polymer 1470 may include performing lamination and/or spin coating. As shown in FIG. 9O, the method 1400 may include patterning the biocompatible polymer 1470 down to the carrier substrate 1471. In some embodiments, the patterning may include dry etching.

As shown in FIG. 9P, the method may include releasing the carrier substrate 1471 from the fifth intermediate structure to create the interconnect band 1452. The interconnect band 1452 including one or more electrode contacts 1453a, 1453b, one or more conductive traces (not shown) and/or one or more microelectronic devices 1420a, 1420b encapsulated in one or more encapsulation material(s) 1480 and one or more biocompatible polymer materials 1470. In some embodiments, the electrode contacts 1453a, 1453b may be configured to be disposed on a surface of the brain to record neural activity therefrom. In some embodiments, the microelectronic devices 1420a, 1420b may include associated analog circuitry for recording and/or at least partially process the analog signals.

In some embodiments, the neural device (e.g., the nodes and the bands) may include an interposer coupled to one or more integrated circuit chips. FIGS. 10A-10F show a method 1500 of manufacturing a lace neural device including an interposer 1570 and one or more integrated circuit chips 1520. The lace neural device may include the interposer 1570 (e.g., a silicon interposer) including an electrode 1553, a conductive trace 1557, and an electrical connector coupled 1518 to the integrated circuit chip 1520 (e.g., an embedded logic die), according to some embodiments.

As shown in FIG. 10A, an interposer 1570 (e.g., silicon interposer) may define a plurality of openings 1555 such that the interposer 1570 forms a lace or mesh structure. In some embodiments, the plurality of openings 1555 may be arranged in a grid configuration. In some embodiments, the interposer 1570 may be a thinned silicon interposer. In some embodiments, at least a portion of the interposer 1570 may be thinned such that the neural device can flex to a curvature of the brain. The interposer 1570 may include at least one electrode 1553 (e.g., a surface electrode such as a μECOG) and at least one conductive trace 1557 disposed on a surface of the interposer 1570. In some embodiments, the at least one electrode 1553 and/or the at least one conductive trace 1557 may be deposited on the interposer 1570 via at least one of physical vapor deposition, chemical vapor deposition, or electroplating. The interposer 1570 may include one or more portions configured to be formed into a node (e.g., hereinafter, “a precursor portion 1510p”). The precursor portion 1510p may include a first electrical contact 1518a disposed on a first side of the interposer 1570, an electrical connection 1518b extending therethrough, and/or a second electrical contact 1518c on a second surface of the interposer 1570 opposite the first electrode contact 1518a (collectively referred to as “the electrical connector 1518”). For example, he electrical connector 1518 may include an electrical contact 1518a disposed on a first surface of the interposer 1570 and an electrical connection 1518b extending from the electrical contact 1518a to a second surface of the interposer 1570 opposite the first surface. To form the node 1510, the steps shown in FIG. 10B-10E may be performed. In some embodiments, the node 1510 may be positioned at an intersection point of the grid configuration. In some embodiments, the node 1510 may be positioned separate from the grid configuration (e.g., adjacent to the grid configuration).

The method 1500 may include disposing an encapsulation material 1580 over at least a portion of the interposer 1570, as shown in FIG. 10B. In some embodiments, the encapsulation material 1580 may be wrapped around the interposer 1570 during fabrication. In some embodiments, the method 1500 may further include removing (e.g., wet etching or dry etching) a portion of the encapsulation material 1580 to define the openings 1555 in the interposer 1570 (not shown). In some embodiments, the etching of the openings 1555 may occur during fabrication and before singulation such that the encapsulation material 1580 is disposed on all sides of the interposer 1570.

As shown in FIG. 10C, the method 1500 may include coupling an integrated circuit chip 1520 (e.g., an embedded logic die) to the second side of the interposer 1570. The integrated circuit chip 1520 may include a silicon integrated circuit (e.g., a silicon ASIC) 1560 including one or more electrical contacts 1562 disposed thereon. The integrated circuit chip 1520 may include a first encapsulation layer 1584 disposed on the first surface thereof. In some embodiments, the first encapsulation layer 1584 may include a second encapsulation material such as, for example, silicon dioxide (SiO2) or SiC. In some embodiments, the SiO2 may be transfer bonded to the integrated circuit chip. In some embodiments, the SiC may be anodically bonded to the integrated circuit chip. The integrated circuit chip 1520 may include a second encapsulation layer 1582 disposed over the first encapsulation layer. In some embodiments, the second encapsulation layer may be wrapped around a portion of the integrated circuit chip 1520 before the integrated circuit chip 1520 is coupled to the interposer 1570. The first encapsulation material and the third encapsulation material may be the same material. In some embodiments, the first encapsulation layer 1582 and the second encapsulation layer 1584 may include different encapsulation materials. In some embodiments, the first encapsulation layer 1584 may include a second encapsulation material, and the second encapsulation layer 1582 may include the first encapsulation material. The integrated circuit chip 1520 may include any suitable circuitry for recording and/or stimulation neural tissue. For example, the integrated circuit chip 1520 may be structurally and/or functionally similar to the analog circuitry as described in FIGS. 3A and 4A.

In some embodiments, the integrated circuit chip 1520 may be coupled to the interposer 1570 and the first side of the interposer 1570. In some embodiments, a first integrated circuit chip may be coupled to the interposer 1570 on the first side thereof and a second integrated circuit chip 1520 may be coupled to the interposer 1570 on the second side thereof. The coupling the integrated circuit chip 1520 to the interposer 1570 may include electrically connecting the electrical contact 1562 of the integrated circuit chip 1520 to the electrical contact 1518a of the interposer 1570 via the electrical connector 1518b. In some embodiments, the electrical connection 1518 may be a through-silicon via (TSV). In some embodiments, the TSV may be coupled to the electrical contact 1562 of the integrated circuit chip 1520 via a bump bond 1519.

As shown in FIG. 10D, the method 1500 may include applying a third encapsulation layer (continuous with the second encapsulation layer in FIG. 10D) on a second surface of the integrated circuit 1520 chip opposite the first surface. The third encapsulation layer may include the first encapsulation material. In some embodiments, the applying third encapsulation layer may include coupling a hard shadow mask 1508 over the electrode 1553 and/or the conductive trace 1557. While the hard shadow mask 1508 is disposed over the electrode(s) 1553 and/or conductive trace(s) 1557, the third encapsulation layer may be disposed on the second side of integrated circuit chip 1520 (e.g., the side facing away from the interposer 1570).

As shown, in FIG. 10E, the hard shadow mask 1508 may be removed leaving the neural device. The neural device may be configured to be disposed on the surface of the brain. In some embodiments, the nodes 1510 (e.g., the node precursor portion 1520p and the integrated circuit chip 1520) may be sufficiently rigid to prevent anisotropic electronic property changes to the integrated circuit during operation thereof.

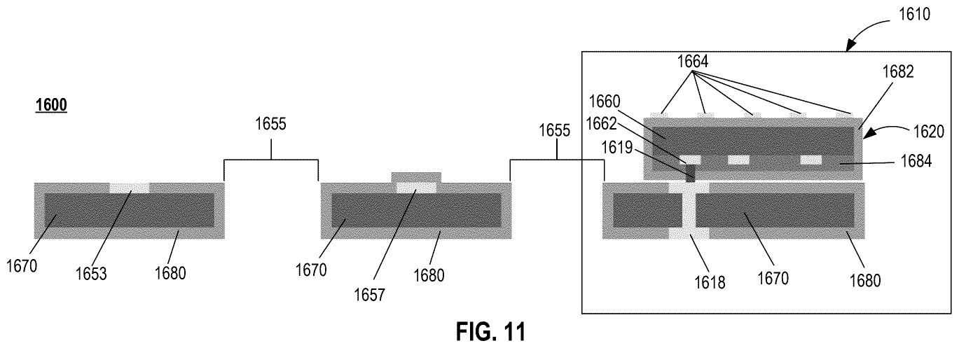

FIG. 11 is a schematic diagram of neural device 1600, according to some embodiments. As shown, the neural device 1600 includes an interposer 1670 (e.g., a thinned silicon interposer) defining a plurality of openings 1655. In some embodiments, the interposer 1670 includes an electrode 1653 and a conductive trace 1657 on a first side of the interposer 1670. The interposer 1670 may be coated in an encapsulation material 1680. In some embodiments, all sides of the interposer 1670 may be at least partially coated in the encapsulation material 1680. The electrode contact 1653 may be at least partially exposed such that the electrode contact 1653 can contact a surface of the brain. In some embodiments, the conductive trace 1657 may be covered to electrically isolate the conductive trace 1657 from the brain. In some embodiments, the conductive trace 1657 may transmit electrical signals between electrode contact(s) 1653 and/or node(s) 1610 of the neural device 1600. In some embodiments, the neural device 1600 may further include a node 1610. The node 1610 may include an electrical contact disposed on a first surface of the node and an electrical connection extending from the electrical contact to a second surface of the node opposite the first surface to form an electrical connector 1618.

In some embodiments, the electrical connector 1618 may electrically connect the interposer 1670 to an integrated circuit chip 1620 via a bump bond 1619. The integrated circuit chip 1620 may be coupled to the interposer 1670 on the first side of the interposer 1670. In some embodiments, the integrated circuit chip 1670 may include a silicon integrated circuit (ASIC) including a plurality of electrical contacts 1662 disposed on a first side thereof. The integrated circuit chip 1670 may further include a first encapsulation layer 1684 disposed on the first side thereof. In some embodiments, the first encapsulation layer 1684 may include a second encapsulation material different than the first encapsulation material. In some embodiments, the integrated circuit chip 1620 further includes a second encapsulation layer 1682 disposed around all sides thereof. The second encapsulation layer 1682 may include the first encapsulation material. In some embodiments, the set of contacts 1662 may be a first set of contacts, and the integrated circuit chip 1620 may include a second set of contacts 1664 disposed on a second side thereof (e.g., a side facing away from the interposer 1670).

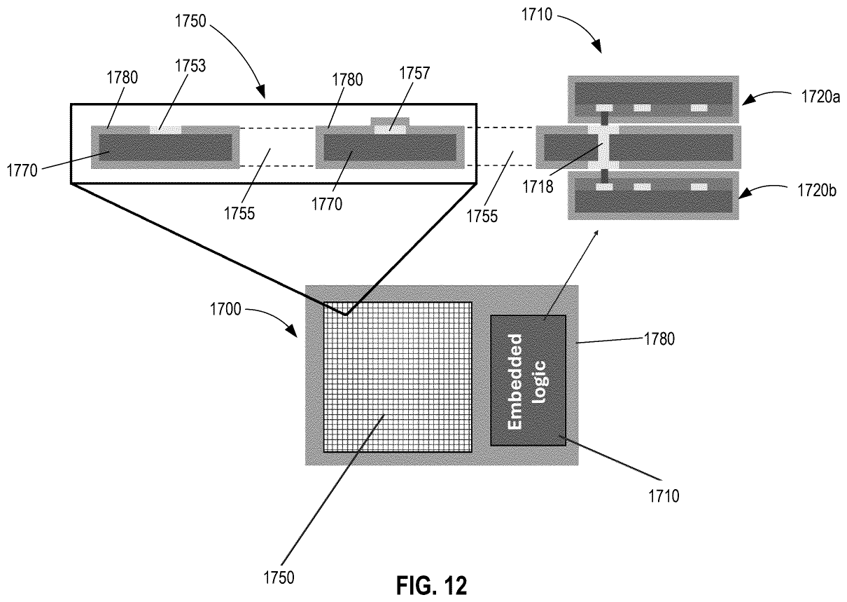

FIG. 12 is a schematic diagram of a neural device 1700 including a grid or mesh configuration and a node 1710 disposed adjacent to. In some embodiments, the grid or mesh may include an interposer 1770 including one or more electrode contacts 1753 and one or more conductive traces 1757. The interposer 1770 may define a plurality of openings 1755 to form the grid configuration. As shown, a portion of the interposer 1770 including the electrode contacts 1753 and the one or more conductive traces 1757 may form the interconnect bands 1750 of the grid or mesh. The node 1710 may include integrated circuit chips 1720a, 1720b coupled to a first side and a second side of the interposer 1770. The node 1710 may be disposed adjacent to the grid or mesh 1750 (e.g., the interconnect bands).

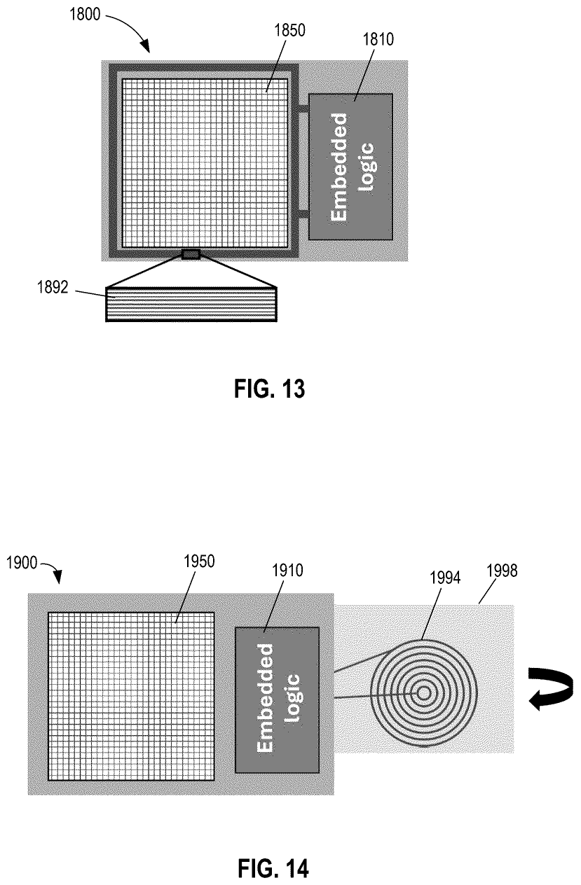

FIG. 13 is a schematic diagram of a neural device 1800 including a peripheral coil 1892 disposed around the grid configuration 1850 and coupled to the node 1810 including one or more integrated circuit chips. The neural device 1800 may be structurally similar to the any of the lace neural devices described in FIGS. 10A-12. FIG. 14 is a schematic diagram of a lace neural device 1900 including a flexible circuit board 1998 (e.g., a flexibly printed circuit board (PCB)) coupled thereto, according to some embodiments. In some embodiments, the flexible circuit board 1998 may include a coil 1994 (e.g., for wireless power and/or communication) and/or any other circuitry (e.g., communication circuitry). In some embodiments, the circuit board 1998 may be coupled to the node 1910 such that analog data collected by the one or more electrode contacts (e.g., electrode contacts in the mesh or grid 1950) can be communicated to the flexible circuit board 1998. In some embodiments, the circuit board 1998 may be configured to wrap over the integrated circuit chip of the neural device 1800, e.g., in the direction indicated by the arrow in FIG. 14. In some embodiments, the encapsulation material may be disposed on at least a portion of the circuit board 1998. For example, the circuit board 1994 may be covered in the encapsulation material to isolate the circuit board 1994 from biological material.

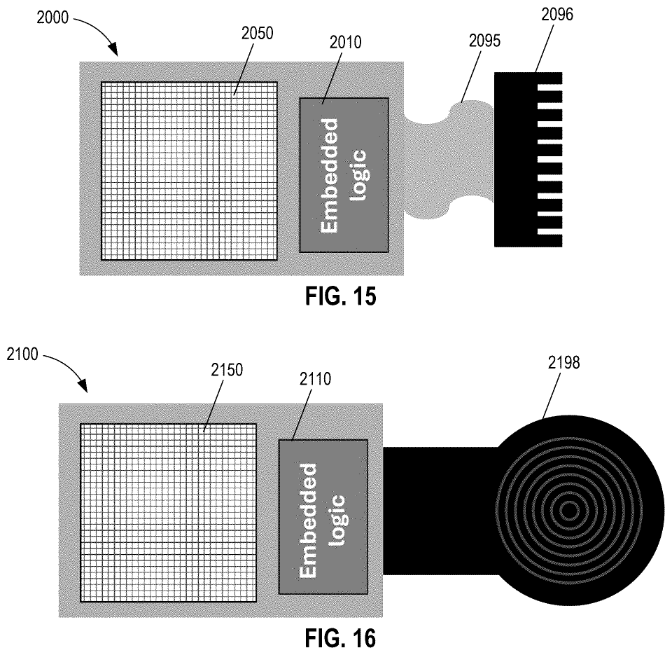

FIG. 15 is a schematic diagram of a neural device 2000 coupled to a wired connector 2096, according to some embodiments. In some embodiments, the neural device 200 may be structurally similar to the any of the lace neural devices described in FIGS. 10A-12. The neural device 2000 may include an interposer defining a plurality of holes in a grid or mesh configuration 2050 and a node 2010 including associated microelectronics (e.g., an integrated circuit chip). In some embodiments, the neural device 2000 may be coupled to a wired connector 2096 through a cable and/or flex circuitry 2095. The wired connector 2096 may be coupleable to an external device (e.g., an implant, further electronic circuitry, a power source, etc.).

FIG. 16 is a schematic diagram of a neural device 2100 coupled to a supplemental device 2198 disposed in a hermetically sealed can with including inductive link, according to some embodiments. In some embodiments, the neural device 2100 may be structurally similar to the any of the neural devices described in FIGS. 10A-12. The neural device 2100 may include an interposer defining a plurality of holes in a grid or mesh configuration 2150 and a node 2110 including associated microelectronics (e.g., an integrated circuit chip). As shown, the neural device 2100 may be coupled to a supplemental device 2198 including a hermetically sealed can including additional electronics. In some embodiments, the hermetic can include an inductive link (e.g., for wireless power and/or communication).

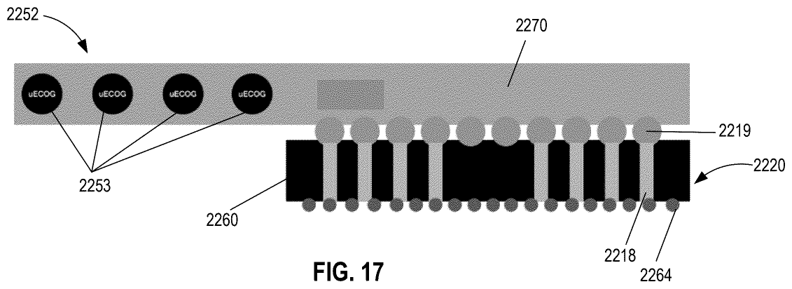

FIG. 17 shows a portion of a neural device including an interconnect band 2252 coupled to associated microelectronics 2220, according to some embodiments. The interconnect band 2252 may include a polymer material 2270 (e.g., LCP, polyimide, and/or silicon). The microelectronics 2320 may include an integrated circuit chip 2260 (e.g., an AMS/ASIC/embedded logic). The integrated circuit chip 2260 may include a first set of electrode contacts 2264 disposed on a first side thereof (e.g., opposite the polymer carrier 2270). The integrated circuit chip 2260 may include a second set of contacts 2219 disposed on a second side opposite the first side. The first set of contacts 2264 and the second set of contacts 2219 may be connected by an electrical connection (e.g., a TSV) 2218 that extends from the first side to the second side. In some embodiments, the second set of contacts 2219 may be coupled to the polymer carrier 2270. As shown, the second set of contacts 2219 can be coupled to the interconnect band 2252 (e.g., analog interconnect band) including one or more surface electrodes (e.g., μECOG). Although not shown, the interconnect band 2252 may include an encapsulation material. In some embodiments, the interconnect band 2252 may include a silicon interposer and an encapsulation material.

In some implementations of one or more embodiments set forth herein, a neural device can further include one or more range extenders, such as a needle structure(s) or other electrically conductive structure(s) that emanate from a surface(s) of the neural device. The range extender(s) may be fabricated, for example, using a wafer-level process, and the range extender(s) may be positioned and/or configured such that they connect directly to an electrode contact(s) of the neural device (e.g., to a lace-like structure thereof).

Various concepts may be embodied as one or more methods, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Put differently, it is to be understood that such features may not necessarily be limited to a particular order of execution, but rather, any number of threads, processes, services, servers, and/or the like that may execute serially, asynchronously, concurrently, in parallel, simultaneously, synchronously, and/or the like in a manner consistent with the disclosure. As such, some of these features may be mutually contradictory, in that they cannot be simultaneously present in a single embodiment. Similarly, some features are applicable to one aspect of the innovations, and inapplicable to others.

In addition, the disclosure may include other innovations not presently described. Applicant reserves all rights in such innovations, including the right to embodiment such innovations, file additional applications, continuations, continuations-in-part, divisionals, and/or the like thereof. As such, it should be understood that advantages, embodiments, examples, functional, features, logical, operational, organizational, structural, topological, and/or other aspects of the disclosure are not to be considered limitations on the disclosure as defined by the embodiments or limitations on equivalents to the embodiments. Depending on the particular desires and/or characteristics of an individual and/or enterprise user, database configuration and/or relational model, data type, data transmission and/or network framework, syntax structure, and/or the like, various embodiments of the technology disclosed herein may be implemented in a manner that enables a great deal of flexibility and customization as described herein.

All definitions, as defined and used herein, should be understood to control over dictionary definitions, definitions in documents incorporated by reference, and/or ordinary meanings of the defined terms.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

While specific embodiments of the present disclosure have been outlined above, many alternatives, modifications, and variations will be apparent to those skilled in the art. Accordingly, the embodiments set forth herein are intended to be illustrative, not limiting. Various changes may be made without departing from the spirit and scope of the disclosure. Where methods and steps described above indicate certain events occurring in a certain order, those of ordinary skill in the art having the benefit of this disclosure would recognize that the ordering of certain steps may be modified and such modification are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

1. An apparatus, comprising:
a biocompatible substrate configured to be disposed on a surface of a brain, the biocompatible substrate including a plurality of nodes and a plurality of connectors interconnecting the plurality of nodes, the biocompatible substrate defining a plurality of openings therein, each of the plurality of nodes and the plurality of connectors including at least one encapsulation material,
the plurality of nodes including:
a first set of nodes, each node from the first set of nodes including (i) an associated set of electrodes from a plurality of electrodes and (ii) an associated analog circuit,
a second set of nodes different than the first set of nodes, each node from the second set of nodes including a digital circuit; and
a third set of nodes different than the first set of nodes and the second set of nodes, each node from the third set of nodes configured to supply power to the first set of nodes and to supply power to the second set of nodes.

2. The apparatus of claim 1, wherein the plurality of electrodes is a first plurality of electrodes, the first set of nodes are interconnected via a subset of connectors from the plurality of connectors, the subset of connectors including a second plurality of electrodes.

3. The apparatus of claim 2, wherein the first plurality of electrodes include an array of depth electrodes and the second plurality of electrodes includes a plurality of surface electrodes.

4. The apparatus of claim 3, wherein the plurality of surface electrodes includes at least one of electrocorticography (ECOG) electrodes or micro-electrocorticography (μECOG) electrodes.

5. The apparatus of claim 3, wherein the first set of nodes is configured to perform at least one of record neural data or stimulate a region of a brain via at least one of the array of depth electrodes or the plurality of surface electrodes.

6. The apparatus of claim 1, wherein the analog circuit of the first set of nodes includes at least one of (i) a temperature sensor, or (ii) a charge pump for generating voltage to stimulate a region of a brain.

7. The apparatus of claim 1, wherein the digital circuit of the second set of nodes is configured to perform node-level signal processing including at least one spike detection, threshold estimation, data compression, or early-stage processing of neural signals recorded by the first set of nodes.

8. The apparatus of claim 1, wherein the digital circuit includes a digital system-on-chip (SoC) including:
a plurality of processors;
a network-on-chip (NoC); and
one or more hardware accelerators configured to execute at least one signal processing algorithm.

9. The apparatus of claim 8, wherein the at least one signal processing algorithm includes at least one of training, spike classification, or determination of spike timing such that the apparatus is configured to perform closed-loop neural modulation.

10. The apparatus of claim 1, wherein the first set of nodes are interconnected via a first subset of connectors from the plurality of connectors configured to transfer analog signals, the first subset of connectors extending along a first axis and a second axis, the apparatus further comprising:
a second subset of connectors extending along a third axis and a fourth axis, the third axis and the fourth axis different than the first axis and second axis such that the first subset of connectors and the second subset of connectors do not physically overlap with one another.

11. The apparatus of claim 1, wherein the plurality of openings are defined by the plurality of connectors such that the biocompatible substrate forms a lattice structure.

12. The apparatus of claim 1, wherein the at least one encapsulation material includes at least one of silicon carbide, parylene-C, silicon dioxide, or polyimide.

13. The apparatus of claim 1, wherein at least some of the plurality of nodes include an integrated circuit (IC) chips, each IC chip being sufficiently rigid to prevent anisotropic electronic property changes during operation thereof.

14. The apparatus of claim 1, wherein each connector from the plurality of connectors is flexible such that the apparatus, when disposed on the surface of the brain of a patient, fits to a curvature of the surface of the brain.

15. The apparatus of claim 14, wherein an area of each of the plurality of nodes is smaller than 1 mm² such that the plurality of nodes do not prevent flexing of the plurality of connectors.

16. The apparatus of claim 10, wherein the apparatus further comprises:
a third subset of connectors, each connector from the first subset of connectors is configured as an analog connector, each connector from the second subset of connectors is configured as a digital band having a first speed of data transmission, and each connector from the third subset of connectors is configured as at least one of a power band or a digital band having a second speed of data transmission.

17. A method, comprising:
coupling a biocompatible polymer substrate to a carrier substrate;
applying a first encapsulation layer over a first surface of the biocompatible polymer substrate, to create a first intermediate structure;
depositing a conductive metal layer on the first encapsulation layer and patterning the conductive metal layer to define one or more electrodes and one or more electrical traces, thereby producing a second intermediate structure that includes the one or more electrodes and the one or more electrical traces;
applying a second encapsulation layer to at least a portion of the second intermediate structure;
coupling a microelectronic device, from a set of at least one microelectronic devices, to each of the one or more electrical traces via the second encapsulation layer, thereby producing a third intermediate structure;
applying a third encapsulation layer on at least a portion of the third intermediate structure, thereby producing a fourth intermediate structure;
applying a layer of polymer material on at least portion of the fourth intermediate structure, thereby producing a fifth intermediate structure; and
releasing the carrier substrate from the fifth intermediate structure to create an interconnect band.

18. The method of claim 17, further comprising:
applying a hard metal mask to at least a portion of the first surface of the biocompatible polymer substrate, the applying the first encapsulation layer including applying the first encapsulation layer to at least a portion of the hard metal mask.

19. The method of claim 17, wherein the layer of polymer material includes at least one of a polyimide or a liquid crystal polymer (LCP).

20. The method of claim 17, wherein at least one of the first encapsulation layer, the second encapsulation layer, or the third encapsulation layer includes silicon carbide (SiC).

21. The method of claim 17, wherein the one or more electrodes include a first material and the one or more electrical traces include a second material different than the first material.

22. An apparatus, comprising:
an interposer defining a plurality of openings therein, the interposer including:
at least one electrode;
a plurality of nodes, each node from the plurality of nodes including (i) an electrical contact disposed on a first surface of the interposer at that node and (ii) an electrical connection extending from the electrical contact to a second surface of the interposer opposite the first surface and at that node; and
a first encapsulation material disposed around at least a portion of the interposer; and
a plurality of integrated circuit chips coupled to the second surface of the interposer and electrically connected to the at least one electrode via the electrical connection, each integrated circuit chip including a second encapsulation material disposed on at least a portion thereof,
the apparatus configured to be disposed on a surface of a brain of a user such that the at least one electrode contacts a surface of the brain of the user.

23. The apparatus of claim 22, wherein the interposer is a thinned silicon interposer, and the electrical connection is a through-silicon via (TSV).

24. The apparatus of claim 22, wherein the plurality of integrated circuit chips is coupled to the interposer using at least one bump bond.

25. The apparatus of claim 22, wherein the plurality of integrated circuit chips includes a plurality of electrode contacts on at least a portion of an outer surface thereof.

26. The apparatus of claim 22, wherein the plurality of openings is arranged in a grid configuration, and at least one node from the plurality of nodes is positioned at an intersection point of the grid configuration.

27. The apparatus of claim 22, wherein the plurality of openings is arranged in a grid configuration, and at least one node from the plurality of nodes is positioned adjacent to the grid configuration.

28. The apparatus of claim 22, wherein at least one of the first encapsulation material or the second encapsulation material includes at least one of Silicon Carbide (SiC) or parylene-C.

29. The apparatus of claim 22, wherein the plurality of integrated circuit chips includes at least one of transfer bonded thermal silicon dioxide (SiO₂) or anodically bonded SiC disposed thereon.

30. The apparatus of claim 22, wherein the interposer includes a polymer.

31. The apparatus of claim 22, wherein the plurality of integrated circuit chips is configured to control electrical energy through the at least one electrode.

32. The apparatus of claim 22, wherein the plurality of integrated circuit chips includes at least one of a mixed signal integrated circuit or an application-specific integrated circuit.

33. The apparatus of claim 22, wherein the interposer further includes at least one electrical trace configured to electrically connect the at least one electrode to the electrical connection.

34. The apparatus of claim 33, wherein the at least one electrode includes a first conductive material and the at least one electrical trace includes a second material different than the first conductive material.
